(19) United States
(12) Patent Application Publication
(10) Pub. No.: US 2007/0083066 A1
(43) Pub. Date: Apr. 12, 2007

(54) Method for the production of 1,7-octadiene and use thereof

(75) Inventors: Volker Bohm, Frankenthal (DE); Michael Roper, Wachenheim (DE); Jurgen Stephan, Mannheim (DE); Regina Benfer, Altrip (DE); Markus Schubert, Ludwigshafen (DE); Jorn Karl, Ludwigshafen (DE); Klaus Ebel, Lampertheim (DE); Oliver Lober, Freimersheim (DE); Martin Volland, Heidelberg (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/572,077

(22) PCT Filed: Sep. 17, 2004
(86) PCT No.: PCT/EP04/10435

§ 371 Date: Mar. 16, 2006

(30) Foreign Application Priority Data
Sep. 25, 2003 (DE) 103 44 690.7

(51) Int. Cl. C07C 45/68; C07C 45/49; C07C 5/08

(52) U.S. Cl. 568/345; 568/451; 585/601

(57) ABSTRACT

The invention relates to a method for the production of 1,7 octadiene by reacting metathesis of cyclohexene with ethylene. The invention also relates to the production of 1,10-decandiol by hydroformulating 1,7 octadiene produced according to said method. The invention further relates to a method for the production of muscone or olefinically unsaturated analogs thereof using 1,10 decandiol which is obtainable in said manner.

The present invention relates to a process for preparing 1,7-octadiene by metathesis of cyclohexene with ethylene. It further relates to processes for preparing 1,10-decanedial or muscone or olefinically unsaturated analogs thereof using 1,7-octadiene prepared in this way.

α,ω-Diolefins are valuable starting materials for many chemical syntheses. Thus, they can be utilized in, for instance, homopolymerization or copolymerization processes to obtain polymers having defined properties. In addition, they serve as starting materials for the preparation of α,ω-diols which can in turn be used in polyester syntheses. Furthermore, the terminal double bonds of the α,ω-diolefins are also suitable for producing a variety of further functions. Thus, for example, double hydroformylation of the terminal double bonds give dialdehydes.

A useful synthetic route to α,ω-diolefins is ring-opening metathesis of cyclic olefins in the presence of ethane (“ethenolysis”). In this type of reaction, the ethenolysis of cyclohexene to form 1,7-octadiene occupies a somewhat exceptional position. Owing to the unfavorable position of the equilibrium in this case, this reaction has hitherto been able to be carried out only in unsatisfactory yields and under conditions which are not very attractive from an economic point of view. Thus, D. L. Crain et al. in Am. Chem. Soc. Div. Petrol. Chem. Prepr. 1972, 17(4), E80-E85, describe the ethenolysis of cyclohexene, which proceeds at a pressure of about 690 bar, a temperature of 125° C. and in the presence of a 9-fold molar excess of ethylene to give a conversion of 6.8%.

DE-A 1 618 760 describes the ethenolysis of cyclic olefins in the presence of suitable transition metal catalysts in general terms. The ethenolysis of cyclohexene in the presence of a catalyst comprising CoO and MoO₃ at a pressure of 52 atm and 125° C. for 3 hours gives 1,7-octadiene in a selectivity of about 23% of theory.

U.S. Pat. No. 3,424,811 describes “disproportionation reactions” of cyclic olefins with acyclic olefins in the presence of supported catalysts comprising Mo or Re and an alkali metal.

DE-A 40 09 910 describes the use of organorhenium oxides on oxidic supports as catalysts for the ethenolytic metathesis of olefinic compounds.

There is therefore still an urgent need for a process which allows 1,7-octadiene to be obtained in an economically satisfactory way by ethenolysis of cyclohexene under conditions which can be implemented on an industrial scale.

We have now found a process for preparing 1,7-octadiene by metathesis of cyclohexene with ethylene, in which the unreacted starting materials and any relatively high-boiling by-products obtained are recirculated in purified form to the reaction mixture.

The process of the invention is suitable for preparing large amounts of 1,7-octadiene in a particularly economical way. Starting materials are the inexpensive and readily available compounds cyclohexene and ethylene. Recirculation of the unreacted purified starting materials and relatively high-boiling by-products enables cyclohexene to be converted into 1,7-octadiene in high yield.

Suitable starting materials for the process of the invention are cyclohexene-comprising hydrocarbon mixtures, in particular ones which comprise mostly cyclohexene. Cyclohexene having a purity of from about 90% to about 99.9%, preferably from about 95% to about 99.9%, particularly preferably from about 98% to about 99.9%, is particularly useful. Preference is given to using a cyclohexene which comprises up to about 1% of polar impurities, in particular oxygen compounds such as cyclohexanol and/or cyclohexanone or peroxides. Particular preference is given to using cyclohexene which is essentially free of polar impurities. In addition, it is advantageously subjected to prepurification by methods which are known per se to those skilled in the art. Thus, for example, it can be passed through a guard bed which comprises high-surface-area aluminum oxides, silica gels, aluminosilicates or molecular sieves. This guard bed serves to dry the starting materials used and to remove substances which can act as catalyst poisons in the subsequent metathesis step. Preferred adsorbent materials are, for example, Selexsorb® CD (from Alcoa Inc.) and CDO and also 3 Å and NaX molecular sieves (13X). Purification is advantageously carried out in drying towers at temperatures and pressures which are selected so that all components are present in the liquid phase.

A further starting material used is ethylene having a purity of generally from about 95 to about 99.99%, preferably from about 98 to about 99.99% or even higher. It can, if required, likewise be prepurified by means of suitable methods, e.g. those described above for the purification of cyclohexene.

The reactants are usually used in a molar ratio of ethylene to cyclohexene of from about 1:1 to about 10:1. Preference is given to a molar ratio of from about 2:1 to about 6:1, particularly preferably from about 2:1 to about 4:1.

The starting substances are introduced in undiluted form or diluted with a diluent which is inert under the reaction conditions selected, for example straight-chain, branched or cyclic hydrocarbons having from 5 to 12 carbon atoms, e.g. cyclohexane, cyclooctane pentane, hexane, heptane, octane, nonane, decane, undecane or dodecane or else higher hydrocarbons or mixtures thereof, into a reactor appropriate for the selected temperature and pressure conditions, for example a pressure gas vessel, a flow tube, a tube reactor, a stirred vessel, a trickle-bed reactor, a bubble column, a cascade of stirred vessels, a loop reactor or a column for reactive distillation. For the purposes of the process of the invention, particular preference is given to a tube reactor in which a catalyst which accelerates the metathesis reaction is present in immobilized form.

The reaction can also be carried out in parallel or alternately in two or more reactors. This procedure allows the process to be continued without interruption when the catalyst systems used in the individual reactors are being regenerated.

Suitable catalysts are ones which accelerate the reaction according to the invention under the chosen conditions. Particularly suitable catalysts are ones which do not catalyze undesirable isomerizations or catalyze them to only a small extent. They usually comprise one or more transition metals of groups VI.b, VII.b or VIII, e.g. Re, W, Mo, Ru, Os, Ta or Nb, either as such or in the form of their compounds or salts. They can in principle be used either in homogeneous form or heterogeneous form, but are preferably used in heterogeneous form. Examples of homogeneous catalysts are the Ru-comprising alkylidene complexes which catalyze alkene metathesis, as are described, for example, in WO 03/011455, WO 00/71554, EP-A 0 921 129 and WO 97/06185. Suitable heterogeneous catalysts are, for example, ones in which the catalytically active substance or compound has been applied in an amount of from about 0.1 to about 20% by weight, preferably from about 1 to about 15% by weight, based on the total weight of the finished catalyst, to a support, preferably an oxidic support such as SiO₂, Al₂O₃ or TiO₂ or a mixed support such as SiO₂/Al₂O₃, B₂O₃/SiO₂/Al₂O₃ or Fe₂O₃/Al₂O₃, preferably Al₂O₃.

A catalyst which is particularly preferred for the purposes of the process of the invention comprises, based on the total weight of the finished catalyst, from about 6 to about 12% by weight of Re₂O₇ applied to Al₂O₃ as support material.

The support material can be used in various forms, but is advantageously used in the form of spheres, extrudates, spirals, rings or trilobes. Among these, preference is given to spheres or extrudates having a diameter or a length of from about 0.5 to about 5 mm, preferably from about 1 to about 2 mm. In a particularly preferred embodiment, extrudates having a length of about 1.5 mm are used as support material.

The catalytically active compound can be applied to the chosen support in various ways which are known per se to those skilled in the art, e.g. by dipping, dry impregnation or vapor deposition. To produce the preferred rhenium catalysts, the support is impregnated with an aqueous ammonium perrhenate solution or preferably with perrhenic acid. Apart from aqueous solutions, it is also possible to use solutions comprising organic solvents such as dioxane, lower alcohols, ketones and/or ethers. After impregnation, which is generally concluded after from about 1 to about 10 hours, the catalysts are dried at temperatures of from about 100 to about 150° C. and subsequently calcined at temperatures above 500° C., preferably above 550° C., for from about 1 to about 5 hours and cooled under a nitrogen atmosphere.

The support can also be pretreated with an inorganic acid, as described in GB 1,216,587. It can also be doped with further metal compounds such as Nb₂O₅, Ta₂O₅, SiO₂, B₂O₅, WO₅, MoO₃, TiO₂ or GeO₂ to increase its activity, as described in EP 0 639 549. The support can also be pretreated thermally as described in EP 1350779.

If the catalysts are not used immediately for the purposes of the process of the invention after they have been produced, it is advisable to calcine them again in the above-described manner before the reaction.

Suitable methods of regenerating the catalyst or catalysts used are in principle the methods known per se to those skilled in the art, as described, for example, in U.S. Pat. No. 3,365,513, EP-A-933 344, U.S. Pat. Nos. 6,281,402, 3,725,496, DE-A-32 29 419, GB 1144085, U.S. Pat. No. 3,726,810, BE 746,924, U.S. Pat. No. 4,072,629, DE-A-19 55 640 and DE-A-34 276 30.

Furthermore, the regeneration of the deactivated catalyst can also be carried out in, for example, 2 stages.

In the 1st stage, the deactivated catalyst is treated with a regeneration gas (regeneration gas 1) at a temperature of from 400 to 800° C.

The regeneration gas 1 is usually a gas selected from the group consisting of nitrogen, noble gases and gas mixtures of nitrogen and noble gas, which may comprise up to 10% of CO₂ or up to 40% of a saturated C₁-C₈-hydrocarbon.

After conclusion of stage 1, stage 2 of the regeneration is commenced. In this stage, the deactivated catalyst which has been pretreated with regeneration gas 1 is treated with a gas mixture comprising an oxygen-comprising gas (regeneration gas 2).

The regeneration gas 2 is preferably pure oxygen or a mixture consisting essentially of from 0.1 to 100% of oxygen, from 50 to 99.9% of a gas selected from the group consisting of nitrogen, noble gases and gas mixtures of nitrogen and noble gas and, if appropriate, up to 10% of CO₂ or up to 40% of a saturated C₁C₈-hydrocarbon.

The regeneration gas 2 is advantageously passed at a gas hourly space velocity of from 50 to 500 liters per kg per hour through a catalyst bed comprising the deactivated catalyst which has been pretreated with regeneration gas. The temperature of the regeneration gas K2 is generally from 350 to 850° C., preferably from 500 to 700° C., particularly preferably 550° C.

The catalyst is introduced in the form of a bed and preferably as fixed-bed catalyst into the tube reactor. The reactants are metered in so that the space velocity over the catalyst is in the range from about 0.2 to about 20 kg/kgh, preferably from about 1 to about 5 kg/kgh.

The pressure in the reactor is chosen in the range from about 20 to about 200 bar, preferably from about 30 to about 120 bar and particularly preferably from about 50 to about 80 bar. The reaction temperature is usually from about 20 to about 250° C., preferably from about 20 to about 130° C. and very particularly preferably from about 30 to about 110° C. The reactants are then normally present in liquid form.

To counter the usually continuous deactivation of the catalyst, the temperature can optionally be increased either continuously or in steps within the abovementioned ranges. In this way, the conversion measured at the reactor outlet can be kept largely constant over time. A constant product stream in the work-up section is then generally ensured.

The outward stream comprising the reaction products is, according to the invention, transferred from the reactor to a first distillation apparatus D1 in which the low-boiling constituents (A) are separated in an appropriate manner from the relatively high-boiling constituents (B). In the present process, the relatively low-boiling constituents (A) in the first distillation stage are predominantly the excess, unreacted ethylene which may comprise small amounts of other compounds and is to be separated off as completely as possible. Owing to the relatively large boiling point difference between the constituents (A) and (B) to be separated, this first distillation stage is advantageously carried out in the form of a depressurization or flash distillation. It is advantageous to select a pressure in the range from about 5 to about 30 bar, preferably from about 5 to about 20 bar, and a temperature in the range from about 10° C to about 50° C., preferably from about 20 to about 40° C. The relatively low-boiling fraction (A) separated off in this way generally comprises from about 90 to about 99.5% by weight, often from about 95 to about 99% by weight, of ethylene. In addition, the relatively low-boiling fraction (A) generally comprises from about 0.5 to about 10% by weight, often from about 1 to about 5% by weight, of unreacted cyclohexene and possibly also small amounts, e.g. from about 0.05 to about 0.5% by weight, of 1,7-octadiene. When using diluents which are inert under the reaction conditions, these can, depending on their boiling point, likewise be separated off either completely or partly. In this case, an altered composition of the fraction (A) has to be taken into consideration.

The relatively low-boiling fraction (A) which has been separated off in this way can be recirculated wholly or partly to the reactor R. It is preferably recirculated as completely as possible, with small amounts being able to be discharged, if appropriate, to avoid accumulation.

The relatively high-boiling constituents (B) of the reaction mixture which are obtained in the first distillation apparatus D1 generally comprise from about 75 to about 80% by weight, often from about 80 to about 90% by weight, of unreacted cyclohexene, from about 5 to about 20% by weight, preferably from about 5 to about 15% by weight, of 1,7-octadiene and additionally from about 1 to about 10% by weight of ethylene. Furthermore, it also comprises the amounts usual in metathesis of high-boiling by-products formed, for example, as a result of subsequent reactions and also the generally predomiriant part of any inert diluents used. The composition indicated above is based on a mixture obtained without use of diluents.

The relatively high-boiling fraction (B) is, according to the invention, transferred to a further distillation apparatus D2. There, its relatively low-boiling constituents (C), hereinafter also referred to as intermediate-boiling fraction, consisting essentially of unreacted cyclohexene, any inert solvent used for diluting the reaction mixture and ethylene which has not been separated off in the first distillation step are separated from the relatively high-boiling fraction (D). Distillation columns known per se to those skilled in the art, for example, are suitable for this purpose. The separation is advantageously carried out at atmospheric pressure or slightly superatmospheric pressure, for example in the range from about 1 to about 10 bar, and using a number of theoretical plates of from about 10 to about 50. The reflux ratio is advantageously in the range from 1.5 to 6.

The intermediate-boiling fraction (C) separated off overhead can likewise be wholly or partly recirculated to the reactor R, if appropriate after complete or partial removal of the inert diluent. It is preferably recirculated as completely as possible, with generally only small amounts being discharged to avoid accumulation.

The relatively high-boiling fraction (D) remaining in the bottom of the distillation apparatus D2 generally comprises a very predominant proportion, i.e. generally above 95% by weight, of 1,7-octadiene together with residues of cyclohexene (normally in the range from about 0.05 to about 0.5% by weight) and the abovementioned high-boiling by-products.

The relatively high-boiling fraction (D) obtained in this way can, according to the invention, be transferred to a further distillation apparatus D3 and there separated into a relatively low-boiling product fraction (P) and a high-boiling by-product fraction (N). Distillation or rectification columns known per se to those skilled in the art, for example, are suitable for this purpose. The separation is generally carried out at atmospheric pressure or, to reduce the operating temperature, under reduced pressure. It is advantageously carried out under atmospheric pressure using from about 10 to 50 theoretical plates and a reflux of ratio of from about 1.5 to 6.

In this way, it is possible to obtain a product fraction (P) which generally comprises from about 98 to about 99.9% by weight of 1,7-octadiene. The product fraction (P) preferably comprises from about 98.5 to about 99.5% by weight of 1,7-octadiene.

The by-product fraction (N) separated off at the bottom characteristically comprises the high-boiling products of the metathesis reaction, e.g. 1,7,13-tetradecatriene (in general from about 60 to 70% by weight), dodecatrienes and small amounts of bicyclic and tricyclic by-products. It is particularly advantageous from an economic point of view to recirculate all or part of this fraction, too, to the reactor R and thus to the production circuit, since, for example, 1,7,13-tetradecatriene can be converted back into 1,7-octadiene by metathesis. To avoid accumulation of undesirable, possibly troublesome by-products, it is advantageous to discharge part of the by-product stream (N) via an outlet E.

The process can be carried out semicontinuously or fully continuously. The economic advantages become particularly apparent when it is carried out fully continuously.

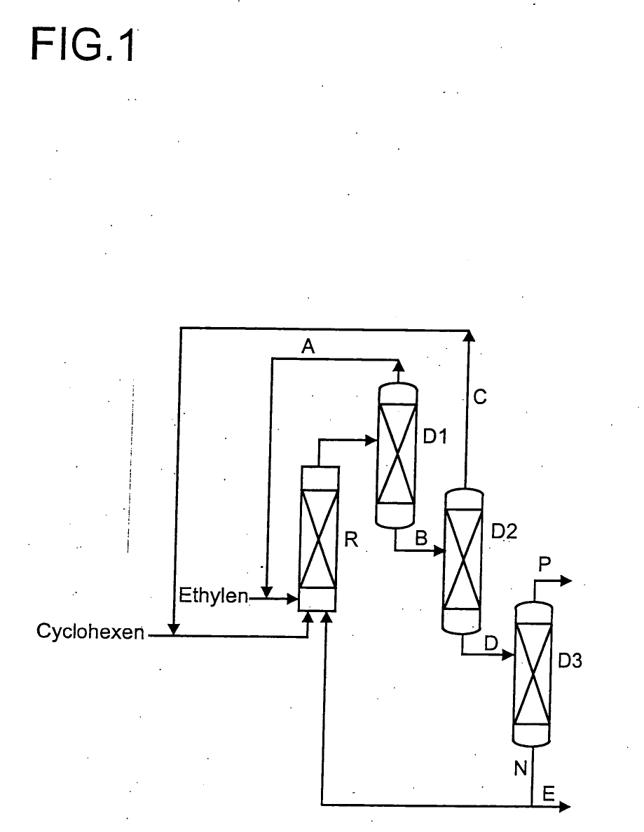

In a preferred embodiment of the process of the invention, an apparatus as shown schematically in FIG. 1 is operated continuously. Accordingly, the reactants ethylene and cyclohexene are introduced in a molar ratio of from about 1:1 to about 6:1 without further dilution into a tube reactor R and there brought into contact with a fixed-bed catalyst comprising 10% by weight of Re₂O₇ on Al₂O₃ in the form of 1.5 mm long extrudates. The temperature in the reactor R is selected so that it is in the range from about 25 to about 130° C., preferably from about 30 to about 100° C. The pressure in the reactor R is advantageously from about 30 to about 120 bar, preferably from about 30 to about 80 bar.

The product stream is subsequently transferred to a flash distillation apparatus D1 and separated at a pressure of from about 5 to about 20 bar and a temperature in the range from about 20 to about 40° C. into a low-boiling fraction (A) and a relatively high-boiling fraction (B).

The low-boiling fraction (A) is recirculated as completely as possible to the feed stream and thus to the reactor R, with only small amounts being discharged to avoid accumulation.

The relatively high-boiling constituents (B) of the reaction mixture, which comprise from about 80 to about 90% by weight of unreacted cyclohexene, from about 7 to about 15% by weight of 1,7-octadiene and from about 1 to about 10% by weight of ethylene, are subsequently transferred to the distillation column D2. There, the intermediate-boiling fraction (C), which consists essentially of unreacted cyclohexene and ethylene which has not yet been separated off, is separated off from the relatively high-boiling fraction (D). The separation is advantageously carried out at atmospheric pressure or slightly superatmospheric pressure, for example in the range from about 1 to about 10 bar, and using a number of theoretical plates of from about 10 to about 50. The reflux ratio is advantageously chosen in the range from 1.5 to 6.

The intermediate-boiling fraction (C) separated off overhead is recirculated as completely as possible to the feed stream and thus to the reactor R. In general, only small amounts are discharged to avoid accumulation.

The fraction (D) which remains in the bottom of the distillation column D2 and generally comprises over 95% by weight of 1,7-octadiene together with residues of cyclohexene (normally in the range from about 0.05 to about 0.5% by weight) is, if a higher purity is desired, transferred to a distillation or rectification column D3 and the relatively low-boiling product fraction (P) is there separated from the high-boiling by-product fraction (N). The separation is generally carried out at atmospheric pressure or, to reduce the operating temperature, under reduced pressure. It is advantageous to set a reflux ratio of from about 1.5 to 6 under atmospheric pressure at from about 10 to 50 theoretical plates.

The by-product fraction (N) separated off at the bottom of the column D3 is likewise wholly or partly recirculated to the reactor R and thus to the production circuit. To avoid accumulation of undesirable, possibly troublesome by-products, part of the by-product stream (N) is advantageously discharged via an outlet E.

It is in this way possible to prepare very pure, i.e. from about 98.5 to about 99.9% strength, 1,7-octadiene on an industrial scale and in an economically attractive way. The desired material obtained in this way is suitable as starting substance or as intermediate for a large number of syntheses of higher value-added products. In particular, it is suitable for the preparation of fine chemicals of all types, e.g. for the preparation of active compounds or additives for pharmaceutical preparations or drugs, cosmetics of any type or foodstuffs or stimulants. The 1,7-octadiene prepared in this way is particularly useful for the preparation of decanedial by double hydroformylation of the two terminal double bonds. Decanedial can serve, inter alia, as starting material for the synthesis of a large number of macrocyclic ketones, e.g. muscone, which are valued fragrances or flavors.

The preparation of the dialdehydes by hydroformylation can be carried out using various systems which are known to those skilled in the art, e.g. Rh/organophosphorus systems or Rh/organopolyphosphorus systems (Appl. Homogeneous Catalysis with Organometallic Compounds, B. Cornielis, W. A. Hermann, VCH, 1996). To achieve high linearities in the Rh-catalyzed hydroformylation, preference is given to using, for example, phosphans, organopolyphosphorus compounds such as chelating phosphanes, chelating phosphites or chelating phosphoramidites as cocatalysts. Examples which may be mentioned are Rh/triphenylphosphine systems (e.g. Falbe, New Synthesis with Carbon Monoxide, Springer-Verlag 1980), Rh/chelating phosphane systems (e.g. WO 01/58589), Rh/chelating biphosphites (e.g. WO 97/20801) or Rh/chelating phosphoramidites (e.g. WO 03/018192, WO 02/83695 and WO 04/026803). WO 04/26803 describes a process for preparing dialdehydes and/or ethylenically unsaturated monoaldehydes by hydroformylation of ethylenically unsaturated compounds.

Hydroformylation catalysts which are suitable for use in the process of the invention are, for example, rhodium complexes having phosphorus ligands of the general formula. I, 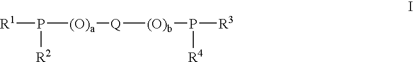
where
Q is a bridging group of the formula 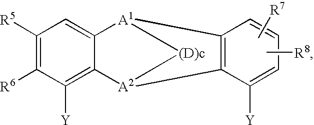
   where
A¹ and A² are each, independently of one another, O, S, SiR^{a}R^{b}, NR^{c} or CR^{d}R^{e}, where
R^{a}, R^{b} and R^{c} are each, independently of one another, hydrogen, alkyl, cycloalkyl, heterocycloalkyl, aryl or hetaryl,
R^{d} and R^{e} are each, independently of one another, hydrogen, alkyl, cycloalkyl, heterocycloalkyl, aryl or hetaryl or together with the carbon atom to which they are bound form a cycloalkylidene group having from 4 to 12 carbon atoms or the group R^{d} together with a further group R^{d} or the group R^{e} together with a further group R^{e} form an intramolecular bridging group D,
D is a divalent bridging group selected from among the groups 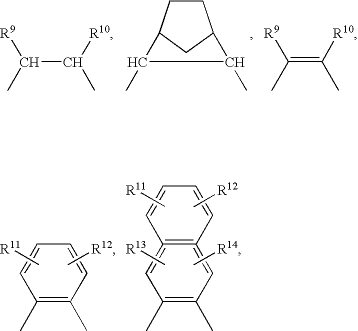
   where
R⁹ and R¹⁰ are each, independently of one another, hydrogen, alkyl, cycloalkyl, aryl, halogen, trifluoromethyl, carboxyl, carboxylate or cyano or are joined to one another to form a C₃-C₄-alkylene bridge,
R¹¹, R¹², R¹³ and R¹⁴ are each, independently of one another, hydrogen, alkyl, cycloalkyl, aryl, halogen, trifluoromethyl, COOH, carboxylate, cyano, alkoxy, SO₃H, sulfonate, NE¹E², alkylene-NE¹E²E³⁺X⁻, acyl or nitro,
is 0 or 1,
Y is a chemical bond,
R⁵, R⁶, R⁷ and R⁸ are each, independently of one another, hydrogen, alkyl, cycloalkyl, heterocycloalkyl, aryl, hetaryl, COOR^{f}, COO⁻M⁺, SO₃R^{f}, SO⁻₃M⁺, NE¹E², NE¹E²E³⁺X⁻, alkylene-NE¹E²E³⁺X⁻, OR^{f}, SR^{f}, (CHR^{g}CH₂O)ₓR^{f}, (CH₂N(E¹))ₓR^{f}, (CH₂CH₂N(E¹))ₓR^{f}, halogen, trifluoromethyl, nitro, acyl or cyano,
   where
R^{f}, E¹, E² and E³ are identical or different radicals selected from among hydrogen, alkyl, cycloalkyl and aryl,
R^{g} is hydrogen, methyl or ethyl,
M⁺ is a cation,
X⁻ is an anion, and
x is an integer from 1 to 120, or
R⁵ and/or R⁷ together with two adjacent carbon atoms of the benzene ring to which they are bound form a fused ring system having 1, 2 or 3 further rings,
a and b are each, independently of one another, 0 or 1,
P is phosphorus and
R¹, R², R³, R⁴ are each, independently of one another, hetaryl, hetaryloxy, alkyl, alkoxy, aryl, aryloxy, cycloalkyl, cycloalkoxy, heterocycloalkyl, heterocycloalkoxy or an NE¹E² group, with the proviso that R¹ and R³ are pyrrole groups bound via the nitrogen atom to the phosphorus P or R¹ together with R² and/or R³ together with R⁴ form a divalent group E which comprises at least one pyrrole group bound via the pyrrole nitrogen atom to the phosphorus atom P and has the formula I
   Py-I-W,
   where
Py is a pyrrole group,
I is a chemical bond or O, S, SiR^{a}R^{b}, NR^{c} or CR^{h}Rⁱ,
W is cycloalkyl, cycloalkoxy, aryl, aryloxy, hetaryl or hetaryloxy, and
R^{h} and Rⁱ are each, independently of one another, hydrogen, alkyl, cycloalkyl, heterocycloalkyl, aryl or hetaryl,
or form a bispyrrole group which is bound via the nitrogen atoms to the phosphorus atom P and has the formula
   Py-I-Py

Preferred phosphoramidite ligands are ligands of the formula Ia 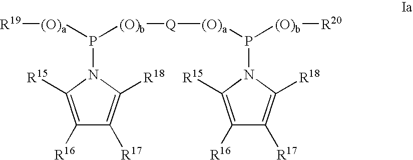
where
R¹⁵, R¹⁶, R¹⁷ and R¹⁸ are each, independently of one another, hydrogen, alkyl, cycloalkyl, heterocycloalkyl, aryl, hetaryl, W′COOR^{k}, W′COO⁻M⁺, W′(SO₃)R^{k}, W′(SO₃)⁻M⁺, W′PO₃(R^{k})(Rⁱ), W′(PO₃)₂⁻(M⁺)₂, W′NE⁴E⁵, W′(NE⁴E⁵E⁶)⁺X⁻, W′OR^{k}, W′SR^{k}, (CHR^{l}CH₂O)_{y}R^{k}, (CH₂NE⁴)_{y}R^{k}, (CH₂CH₂NE⁴)_{y}R^{k}, halogen, trifluoromethyl, nitro, acyl or cyano,
   where
W′ is a single bond, a heteroatom or a divalent bridging group having from 1 to 20 bridge atoms,
R^{k}, E⁴, E⁵, E⁶ are identical or different radicals selected from among hydrogen, alkyl, cycloalkyl and aryl,
R^{l} is hydrogen, methyl or ethyl,
M⁺ is a cation equivalent,
X⁻ is an anion equivalent and
y is an integer from 1 to 240,
   where two adjacent radicals R¹⁵, R¹⁶, R¹⁷ and R¹⁸ together with the carbon atoms of the pyrrole ring to which they are bound may also form a fused ring system having 1, 2 or 3 further rings,
   with the proviso that at least one of the radicals R¹⁵, R¹⁶, R¹⁷ or R¹⁸ is not hydrogen and that R¹⁹ and R²⁰ are not joined to one another,
R¹⁹ and R²⁰ are each, independently of one another, cycloalkyl, heterocycloalkyl, aryl or hetaryl,
   a and b are each, independently of one another, 0 or 1,
P is a phosphorus atom,
Q is a bridging group of the formula
   where
A¹ and A² are each, independently of one another, O, S, SiR^{a}R^{b}, NR^{c} or CR^{d}R^{e}, where
R^{a}, R^{b} and R^{c} are each, independently of one another, hydrogen, alkyl, cycloalkyl; heterocycloalkyl, aryl or hetaryl,
R^{d} and R^{e} are each, independently of one another, hydrogen, alkyl, cycloalkyl, heterocycloalkyl, aryl or hetaryl or together with the carbon atom to which they are bound form a cycloalkylidene group having from 4 to 12 carbon atoms or the group R^{d} together with a further group R^{d} or the group R^{e} together with a further group R^{e} form an intramolecular bridging group D,
D is a divalent bridging group selected from among the groups 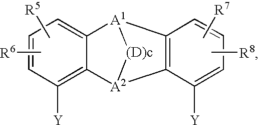 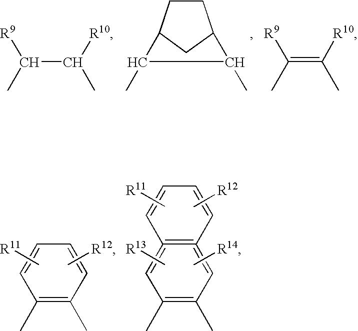
   where
R⁹ and R¹⁰ are each, independently of one another, hydrogen, alkyl, cycloalkyl, aryl, halogen, trifluoromethyl, carboxyl, carboxylate or cyano or are joined to one another to form a C₃-C₄-alkylene bridge,
R¹¹, R¹², R¹³ and R¹⁴ are each, independently of one another, hydrogen, alkyl, cycloalkyl, aryl, halogen, trifluoromethyl, COOH, carboxylate, cyano, alkoxy, SO₃H, sulfonate, NE¹E², alkylene-NE¹E²E³⁺X⁻, acyl or nitro,
c is 0 or 1,
R⁵, R⁶, R⁷ and R⁸ are each, independently of one another, hydrogen, alkyl, cycloalkyl, heterocycloalkyl, aryl, hetaryl, COOR^{f}, COO⁻M⁺, SO₃R^{f}, SO⁻₃M⁺, NE¹E², NE¹E²E³⁺X⁻, alkylene-NE¹E²E³⁺X⁻, OR^{f}, SR^{f}, (CHR^{g}CH₂O)ₓR^{f}, (CH₂N(E¹))ₓR^{f}, (CH₂CH₂N(E¹))ₓR^{f}, halogen, trifluoromethyl, nitro, acyl or cyano,
   where
R^{f}, E¹, E² and E³ are identical or different radicals selected from among hydrogen, alkyl, cycloalkyl and aryl,
R^{g} is hydrogen, methyl or ethyl,
M⁺ is a cation,
X⁻ is an anion, and
x is an integer from 1 to 120, or
R⁵ and/or R⁷ together with two adjacent carbon atoms of the benzene ring to which they are bound form a fused ring system having 1, 2 or 3 further rings.

Such ligands are subject matter of WO 02/083695, which is hereby fully incorporated by reference and in which the preparation of these ligands is also described. Preferred ligands from this class are, for example, the following compounds, with this listing being merely for the purposes of illustration and not having any restrictive character in respect of the ligands which can be employed. 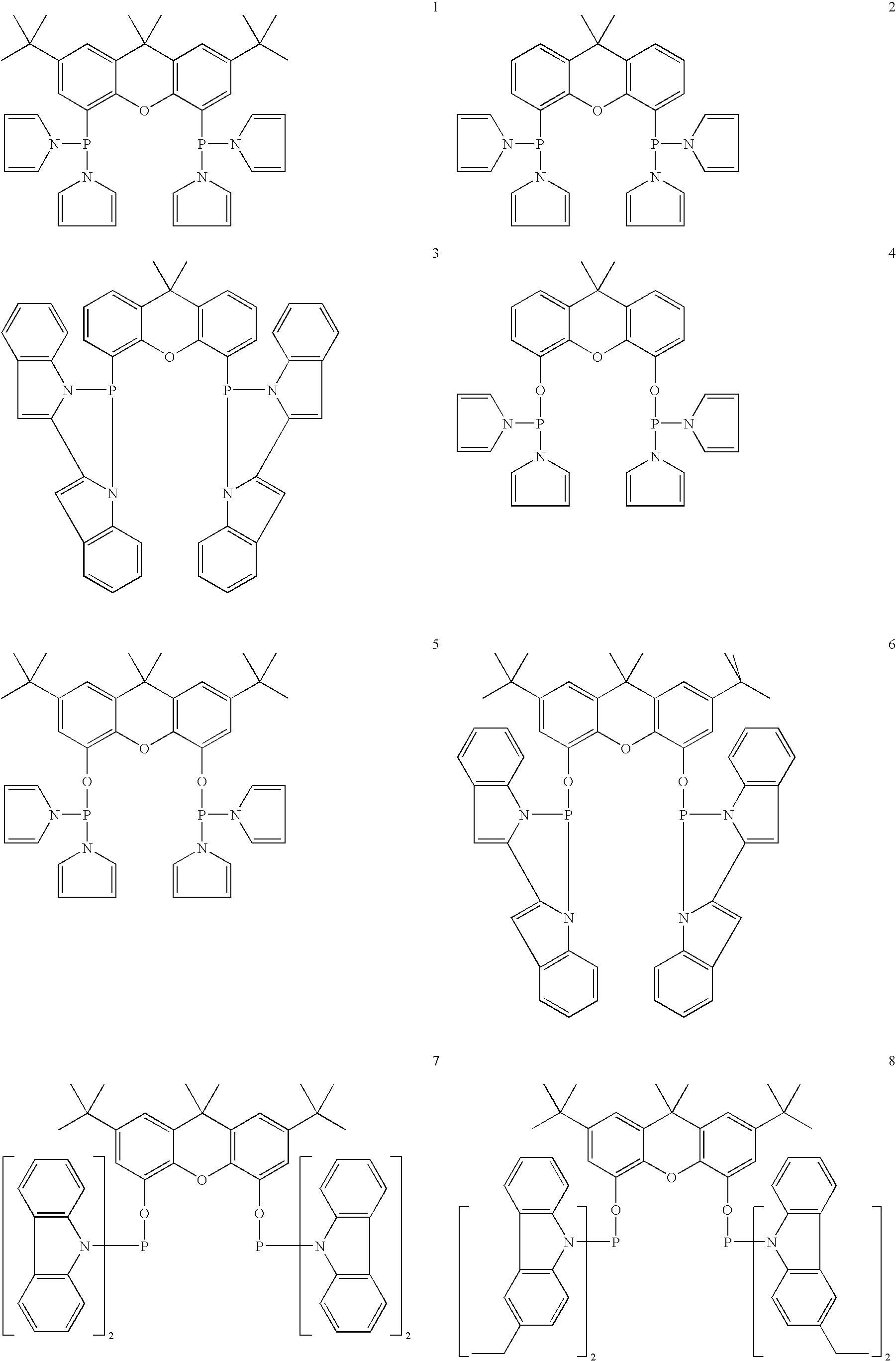 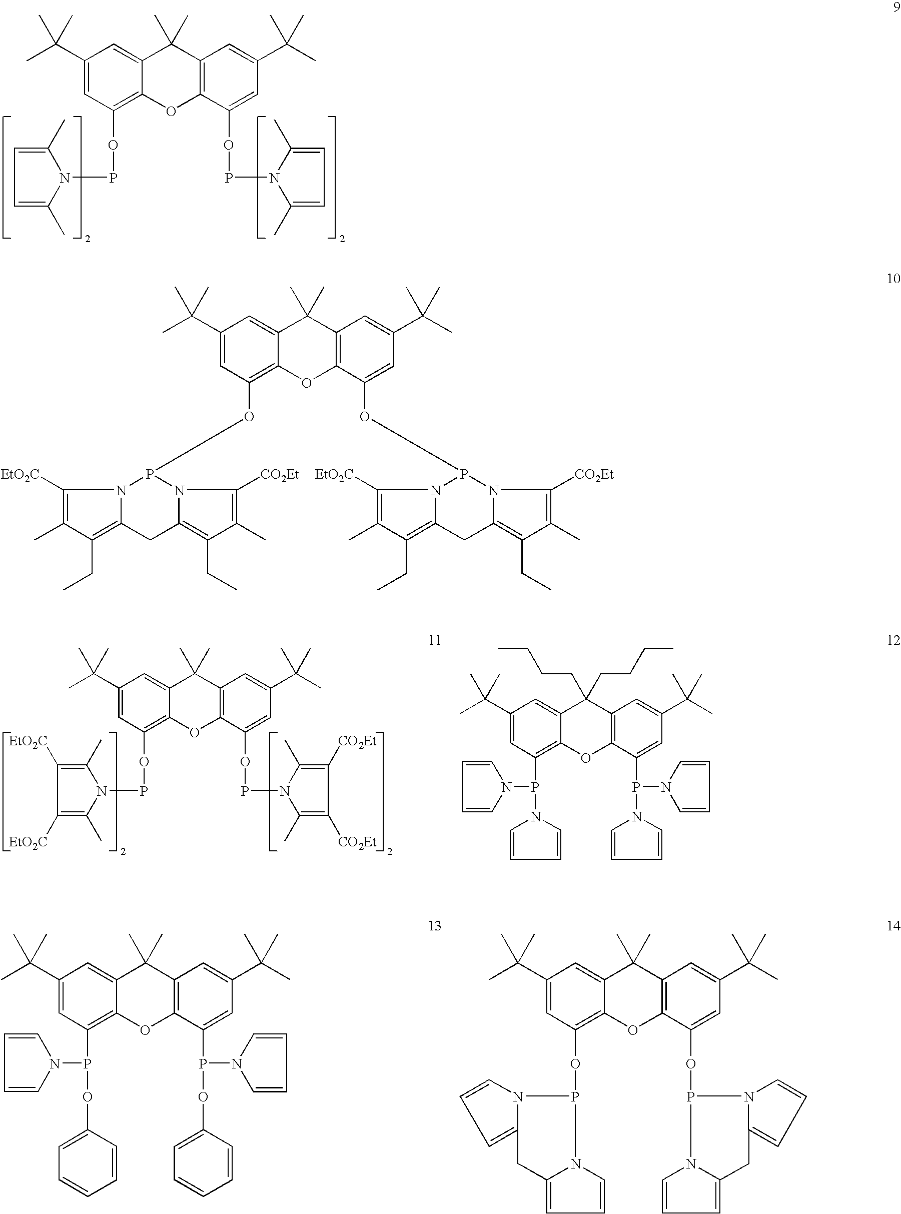 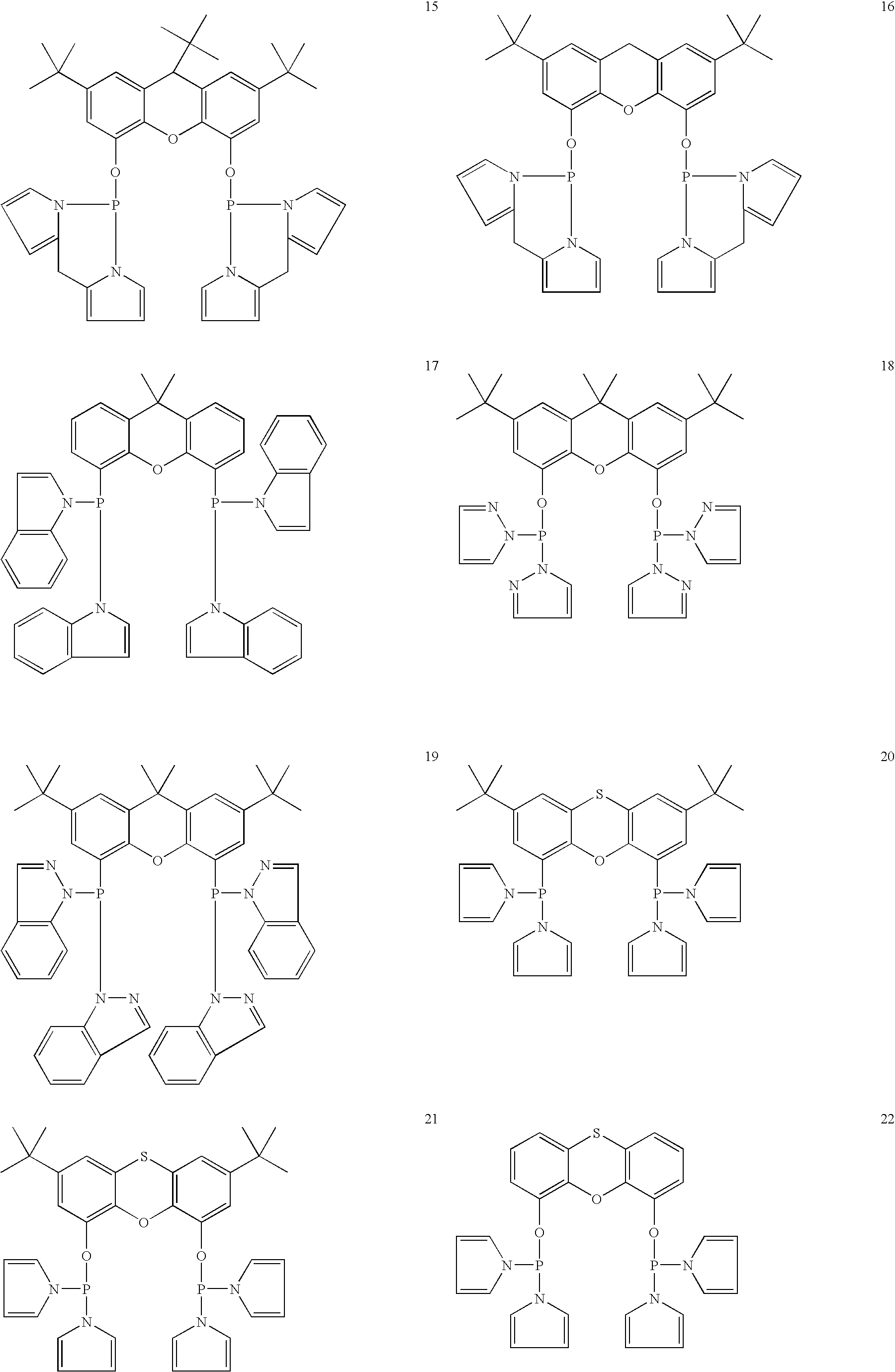 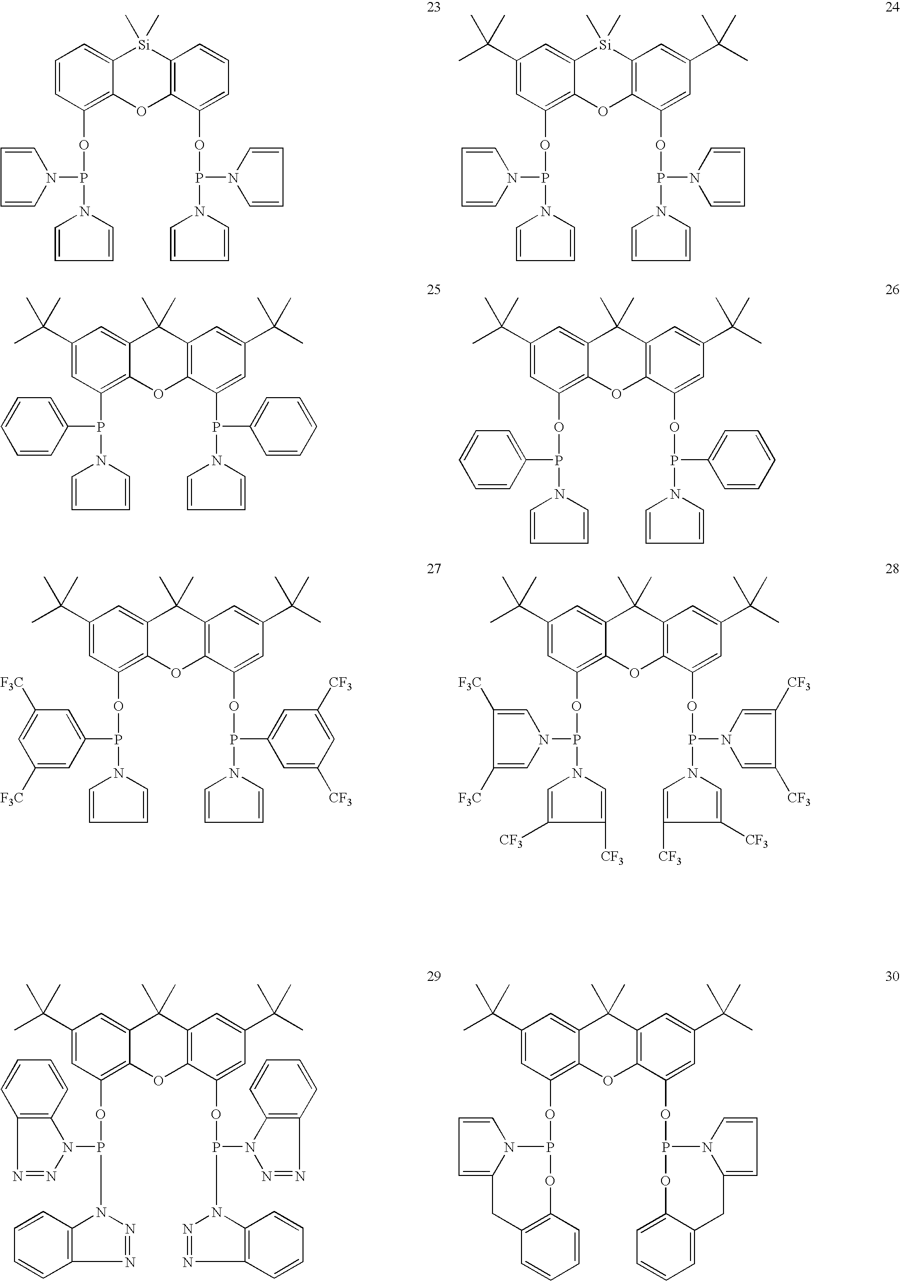 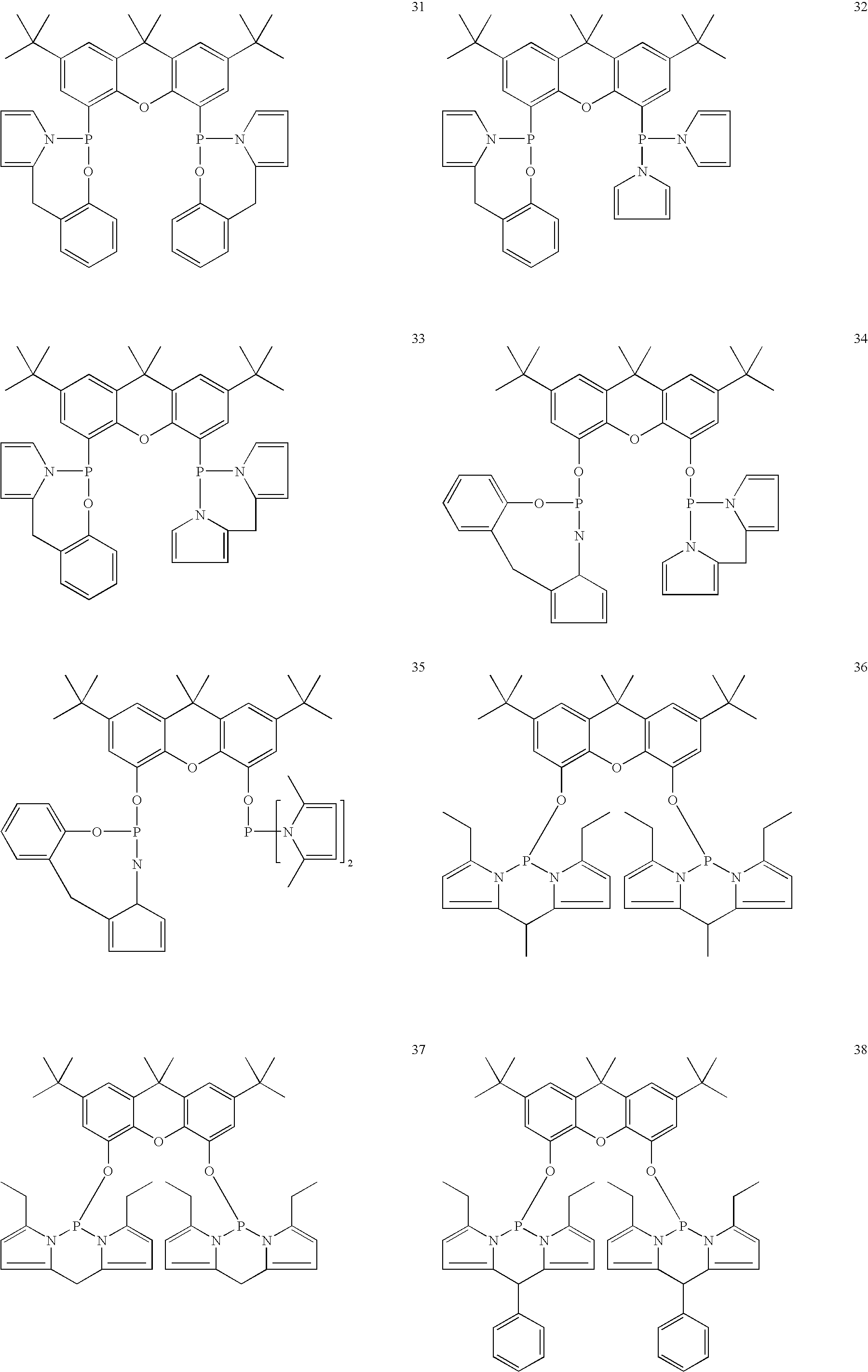 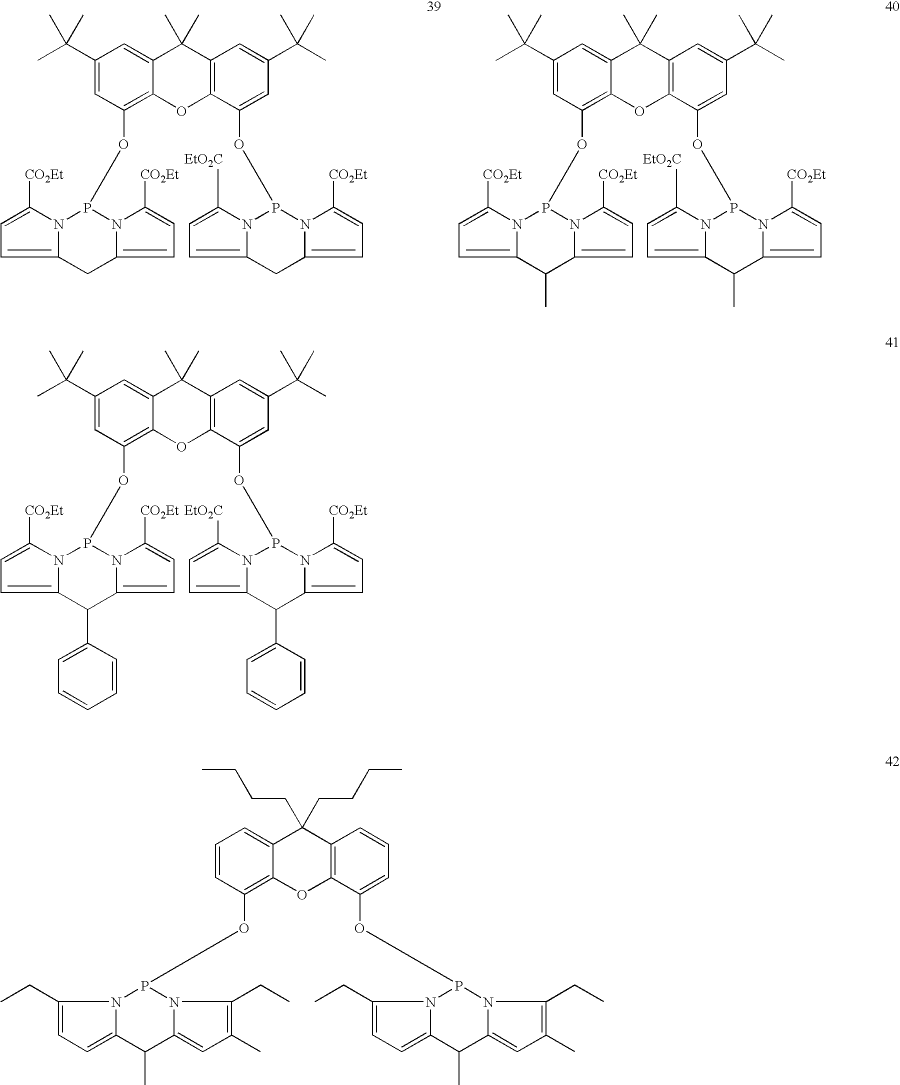 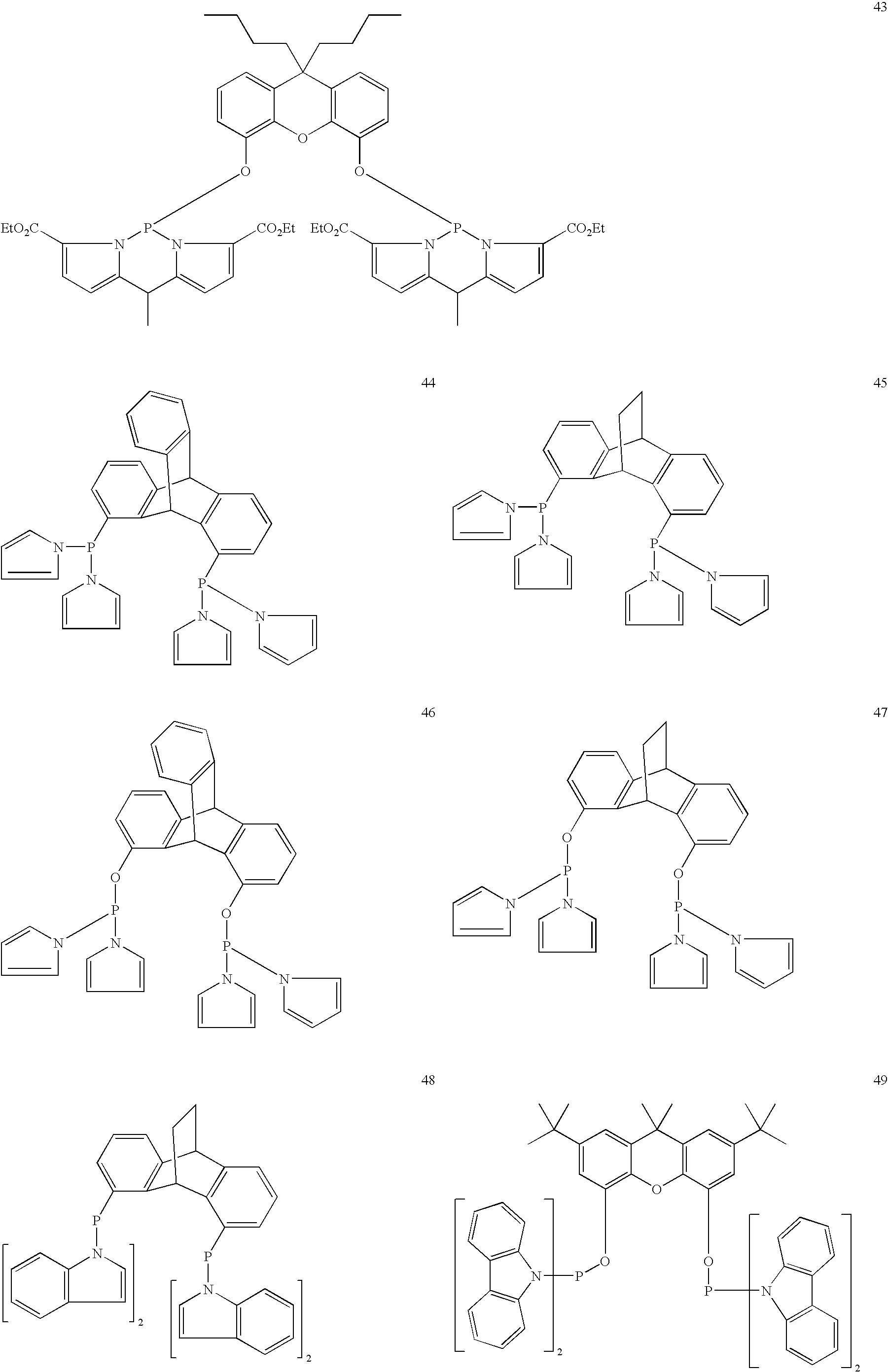 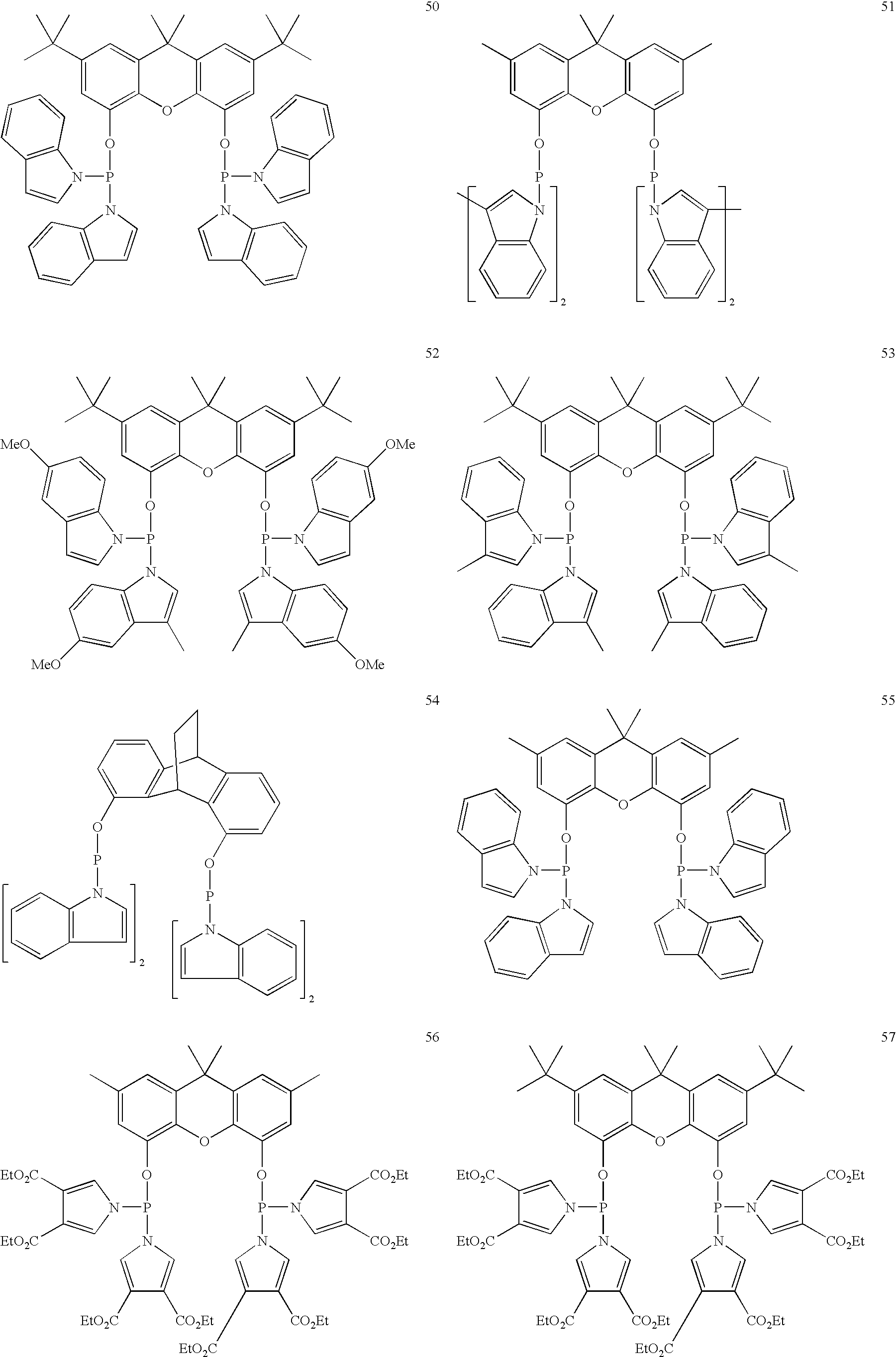
Et: ethyl
Me: methyl

Further suitable phosphorus ligands are diphosphanes and diphosphinites as are described, for example, in WO 01/58589. The following diphosphanes and diphosphinites may be mentioned by way of example:

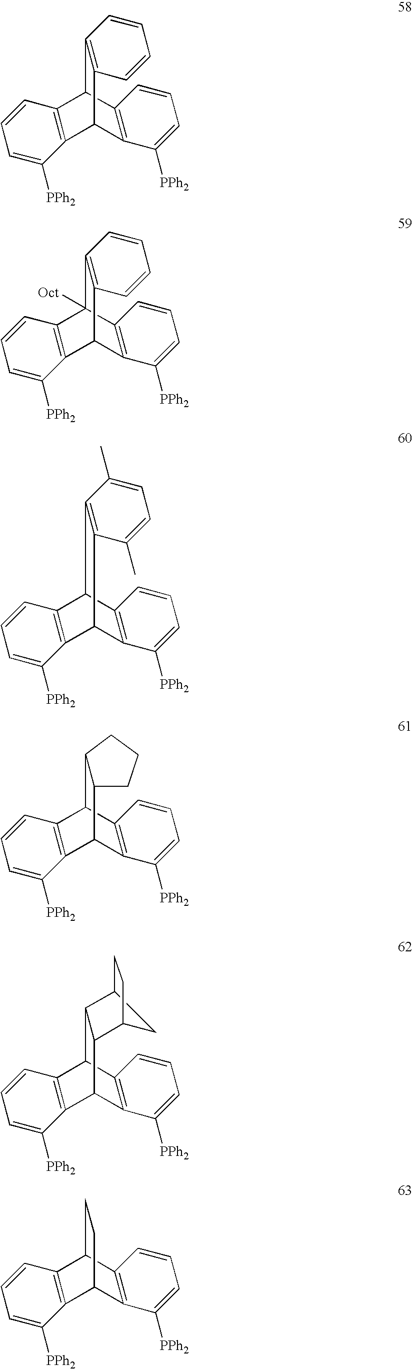

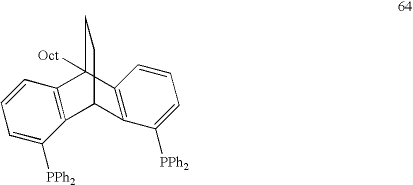

Further ligands which are suitable for the purposes of the process of the invention are the ligands described in WO 95/30680, for example:

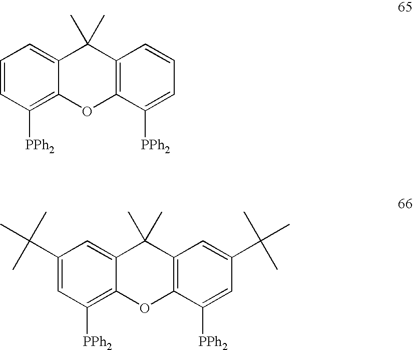

Further suitable phosphoramidite ligands for hydroformylation using rhodium complexes as catalysts are the phosphoramidite ligands described in WO 98/19985 and WO 99/52632, having 2,2′-dihydroxy-1,1′-biphenylene or 2,2′-dihydroxy-1,1′-binaphthylene bridging groups which bear heteroaryl groups such as pyrrolyl or indolyl groups bound via the nitrogen atom to the phosphorus atom, for example the ligands:

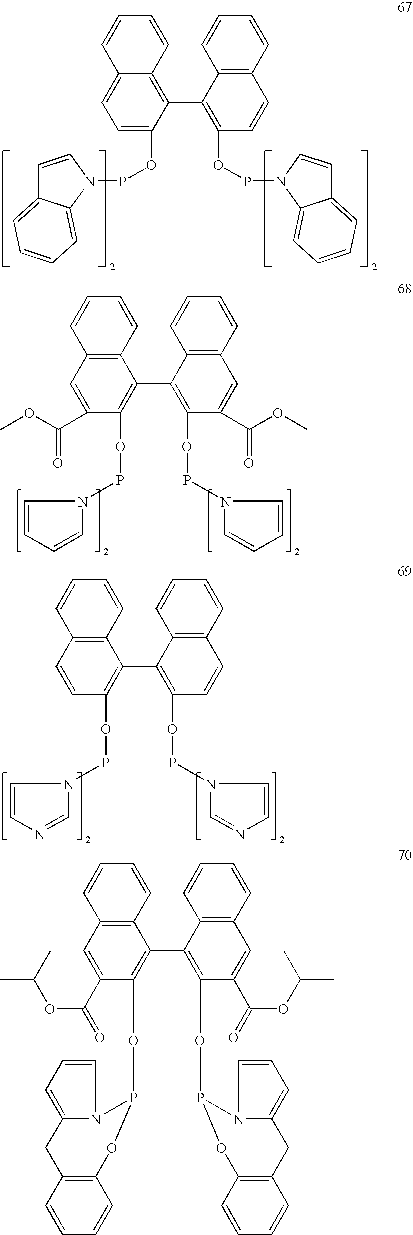

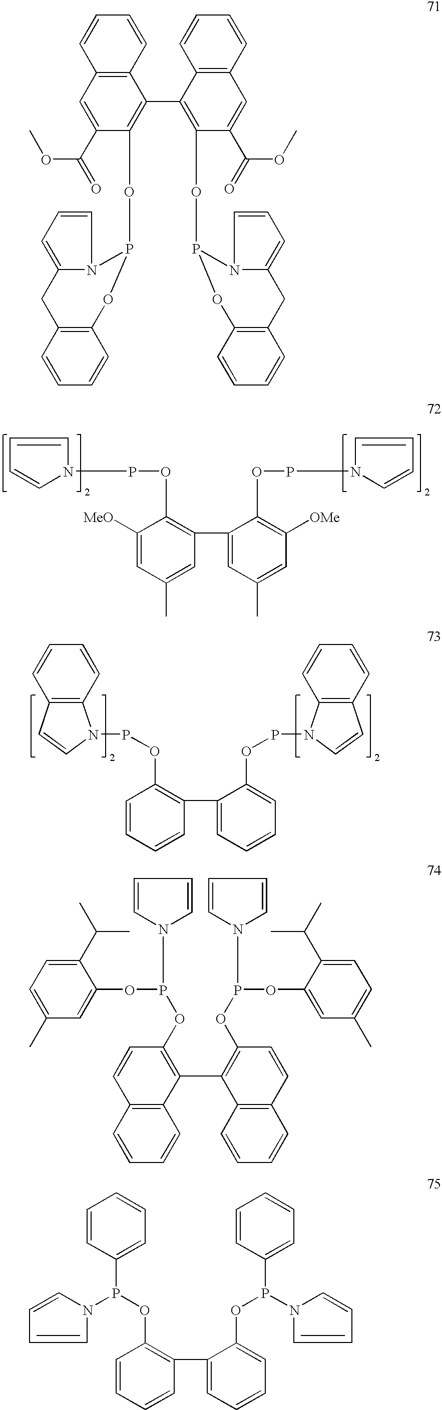

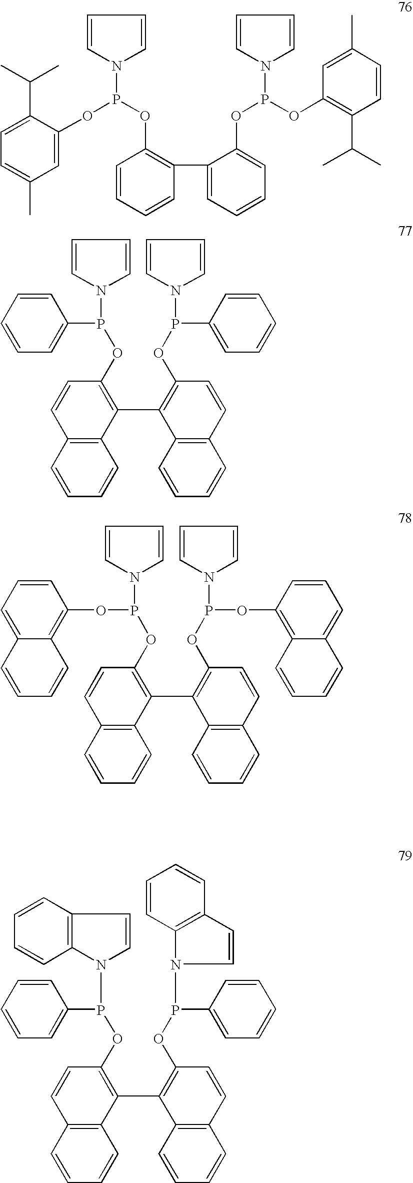

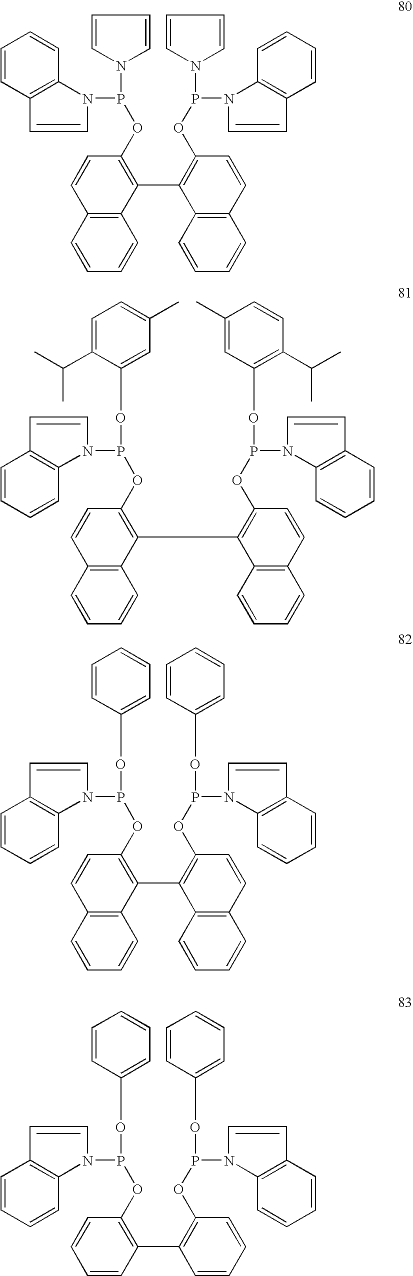

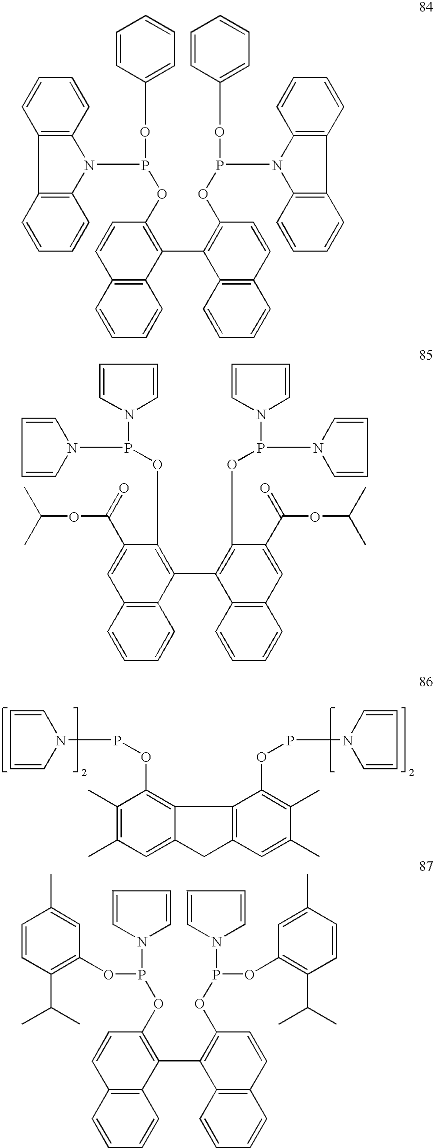

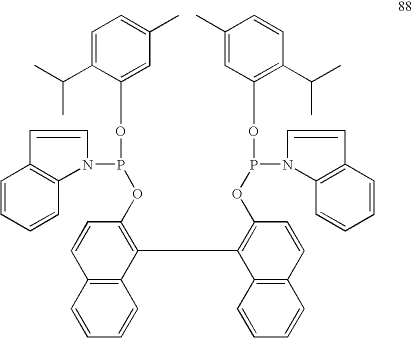

The 1,1′-biphenylene or 1,1′-binaphthylene bridging groups of these ligands can be additionally bridged via the 1,1 positions by a methylene (CH₂—), a 1,1-ethylene (CH₃-CH<) or a 1,1-propylene (CH₃-CH₂-HC<) group.

Further suitable phosphinite ligands for hydroformylation using rhodium complexes as catalysts are, inter alia, the ligands described in WO 98/19985, for example

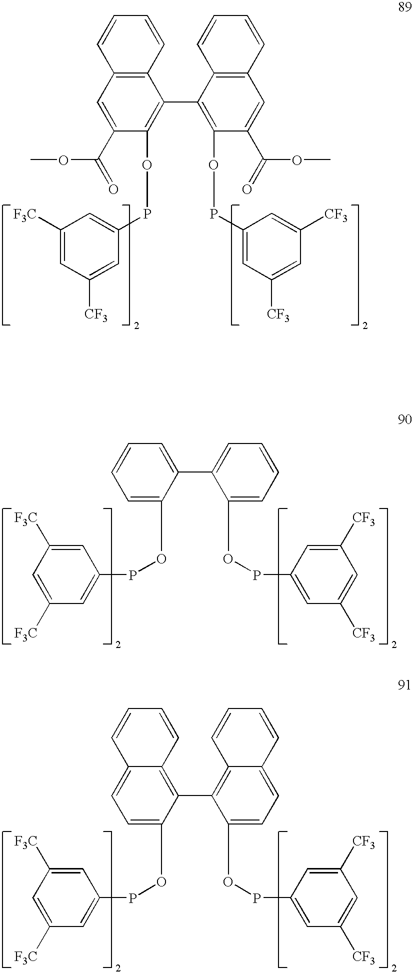

Further suitable ligands for hydroformylation using rhodium complexes as catalysts are phosphite and phosphonite ligands as are described, for example, in WO 01/58589. Merely for the purposes of illustration, the following ligands may be mentioned by way of example:

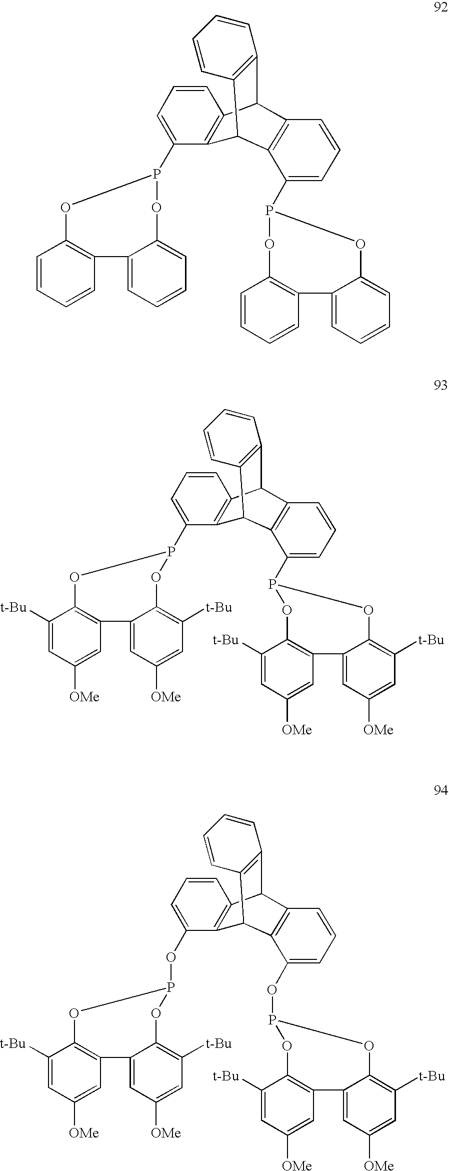

Other well-suited ligands for hydroformylation using rhodium complexes as catalysts are phosphine ligands having the xanthenyl-bis-phosphoxanthenyl skeleton, as are described, for example, in WO 02/068371 and EP-A 982314. Merely for the purposes of illustration, some of these ligands are shown by way of example below.

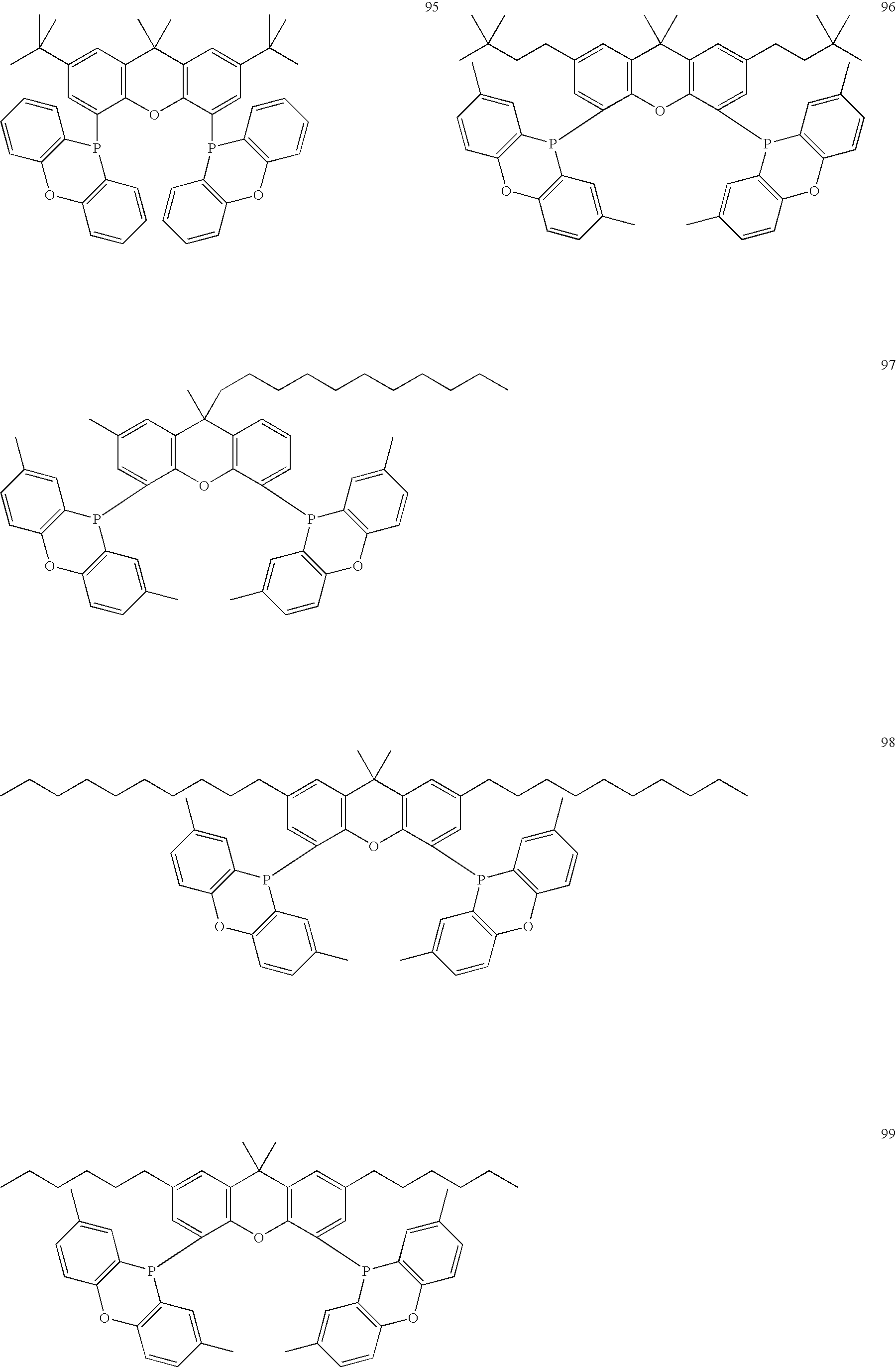

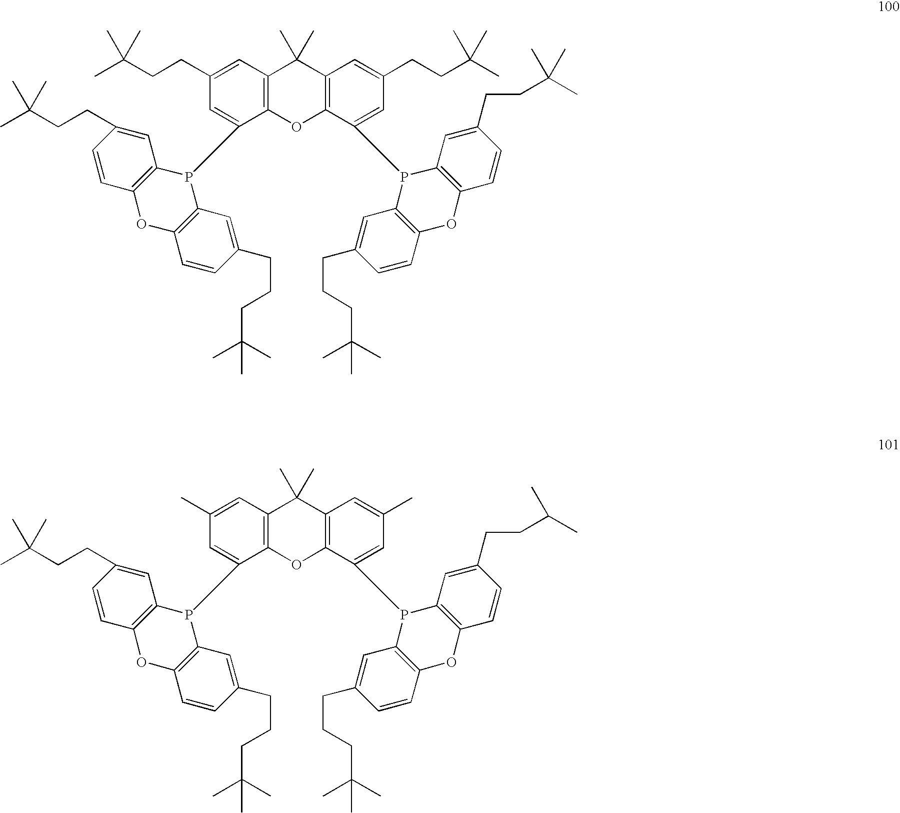

Suitable chelating phosphite ligands for hydroformylation using rhodium complexes of these ligands as catalysts are, for example, those of the general formulae II, III and IV 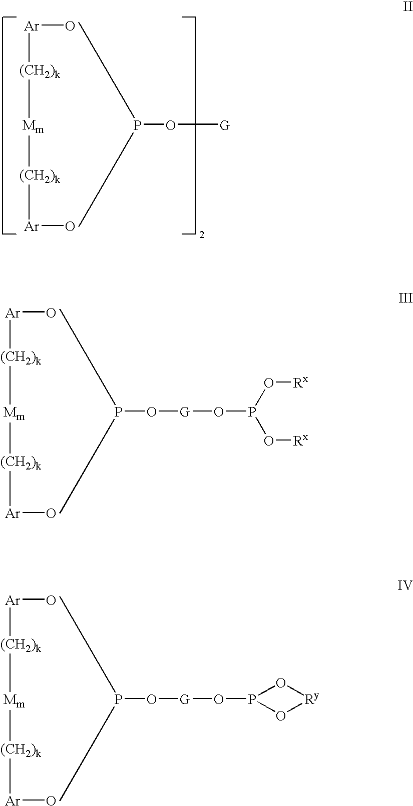
where G is a substituted or unsubstituted divalent organic bridging group having from 2 to 40 carbon atoms, M is a divalent bridging group selected from among —C(R^{w})₂—, —O—, —S—, NR^{v}, Si(R^{t})₂— and —CO—, where the groups R^{w} are identical or different and are each hydrogen, an alkyl group having from 1 to 12 carbon atoms, a phenyl, tolyl or anisyl group, the groups R^{v} are each hydrogen or a substituted or unsubstituted hydrocarbon group having from 1 to 12 carbon atoms, the groups R^{t} are identical or different and are each hydrogen or a methyl group, m is 0 or 1, the groups are identical or different and are each an unsubstituted or substituted aryl group, the index k is 0 or 1, the groups R^{x} are identical or different and are unsubstituted or substituted monovalent alkyl or aryl groups and R^{y} is a divalent organic radical selected from among unsubstituted or substituted alkylene, arylene, arylene-alkylene-arylene and bisarylene groups. Merely for the purposes of illustration but without any restrictive character, the following chelating phosphate ligands which can be used in the process of the invention may be mentioned by way of example: 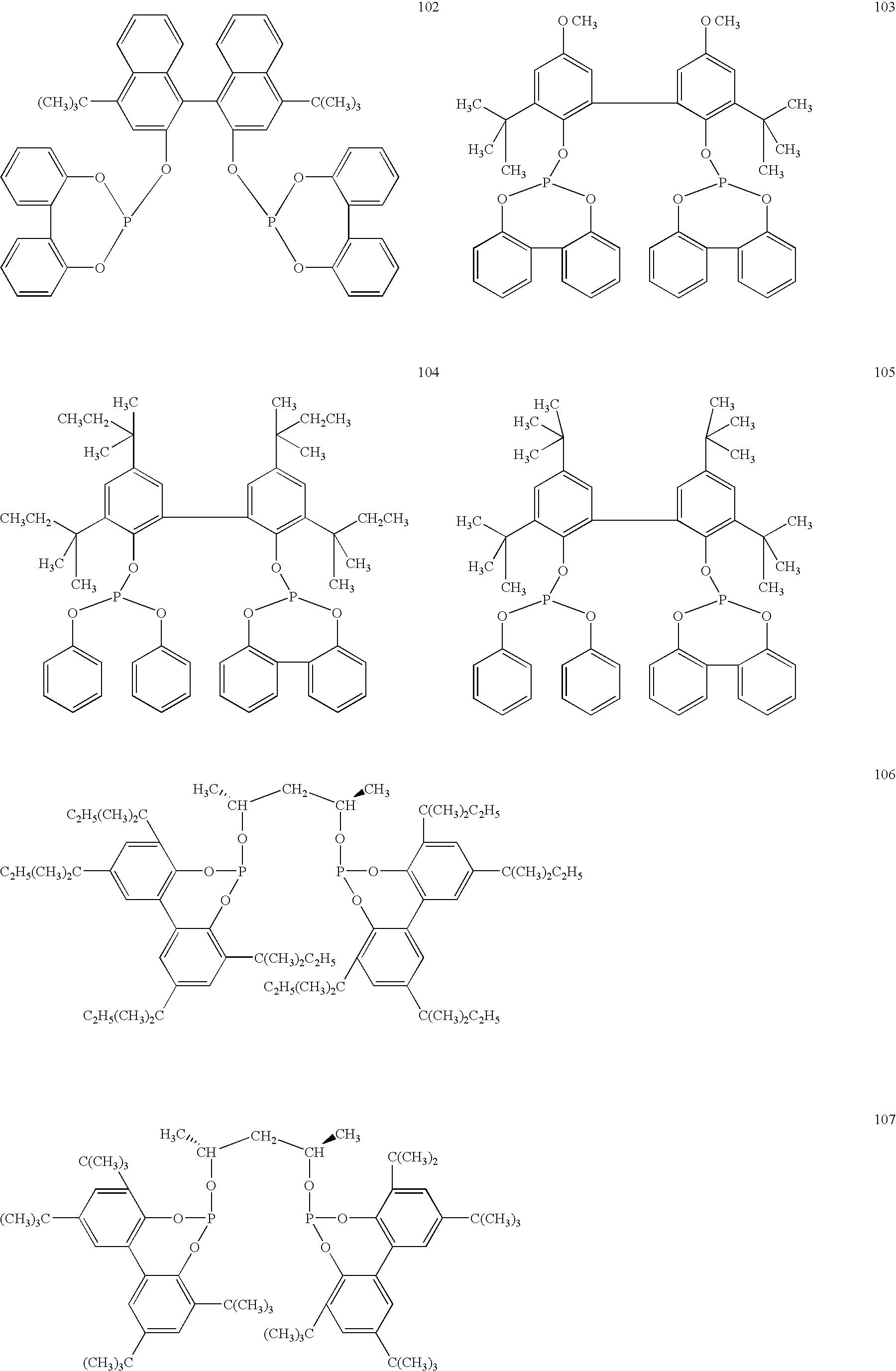 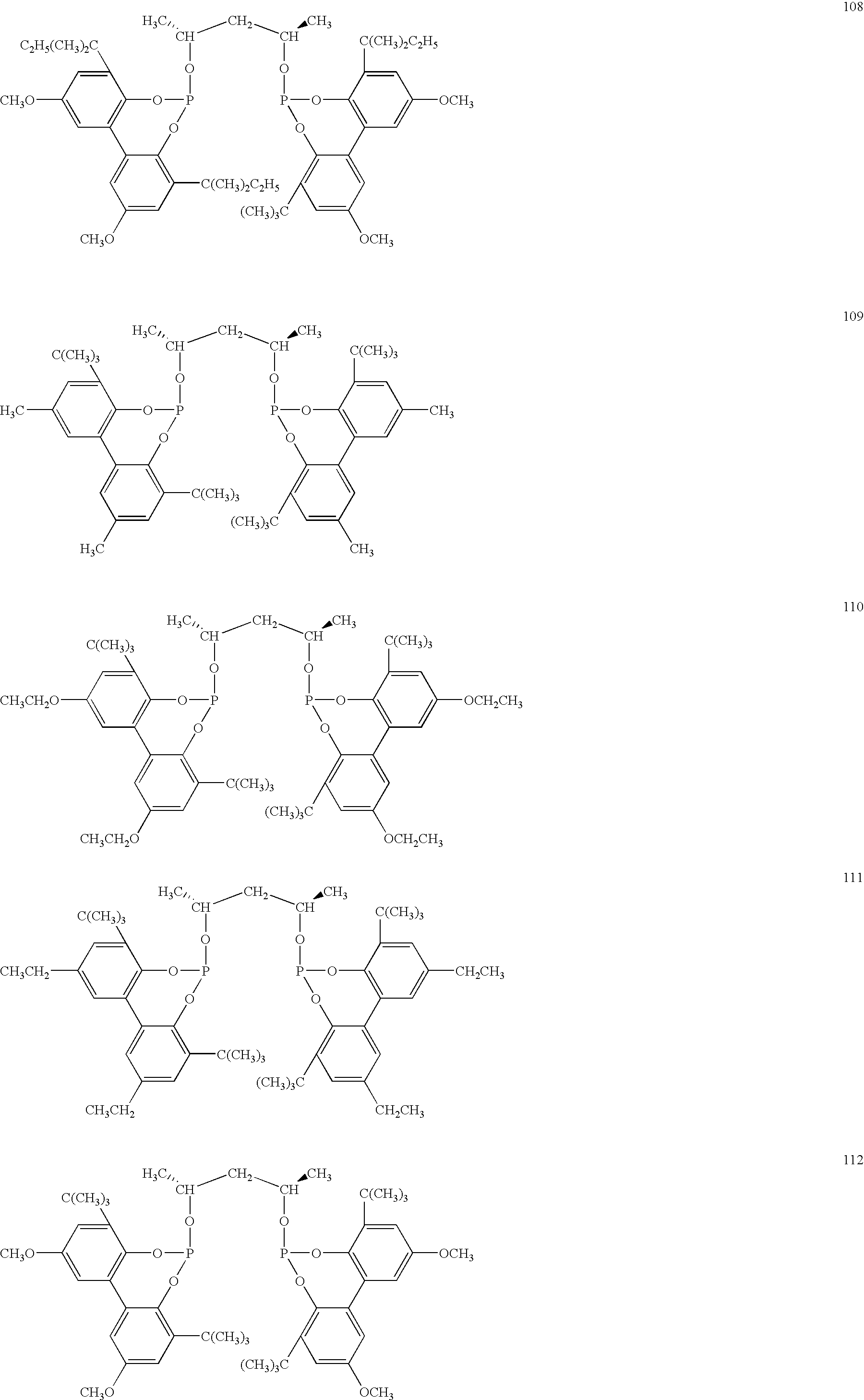 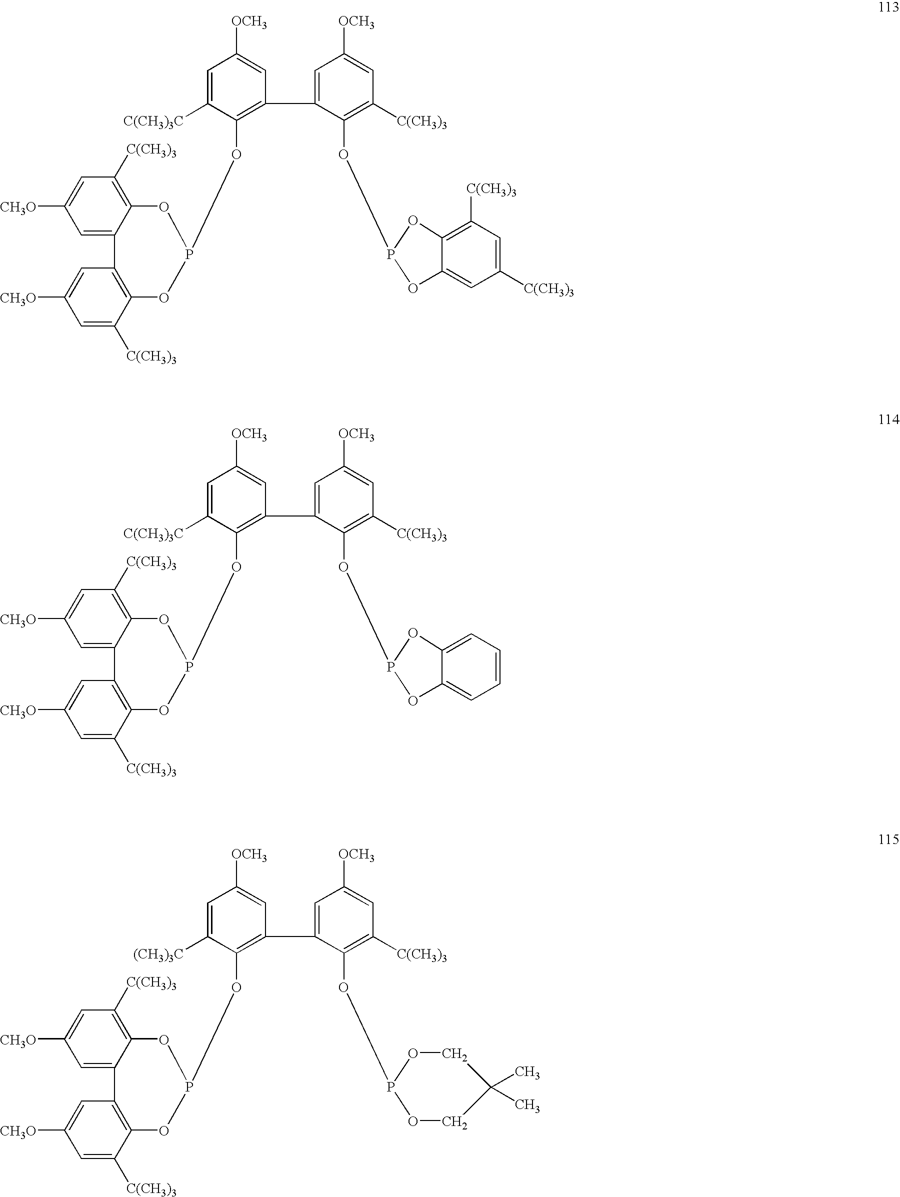 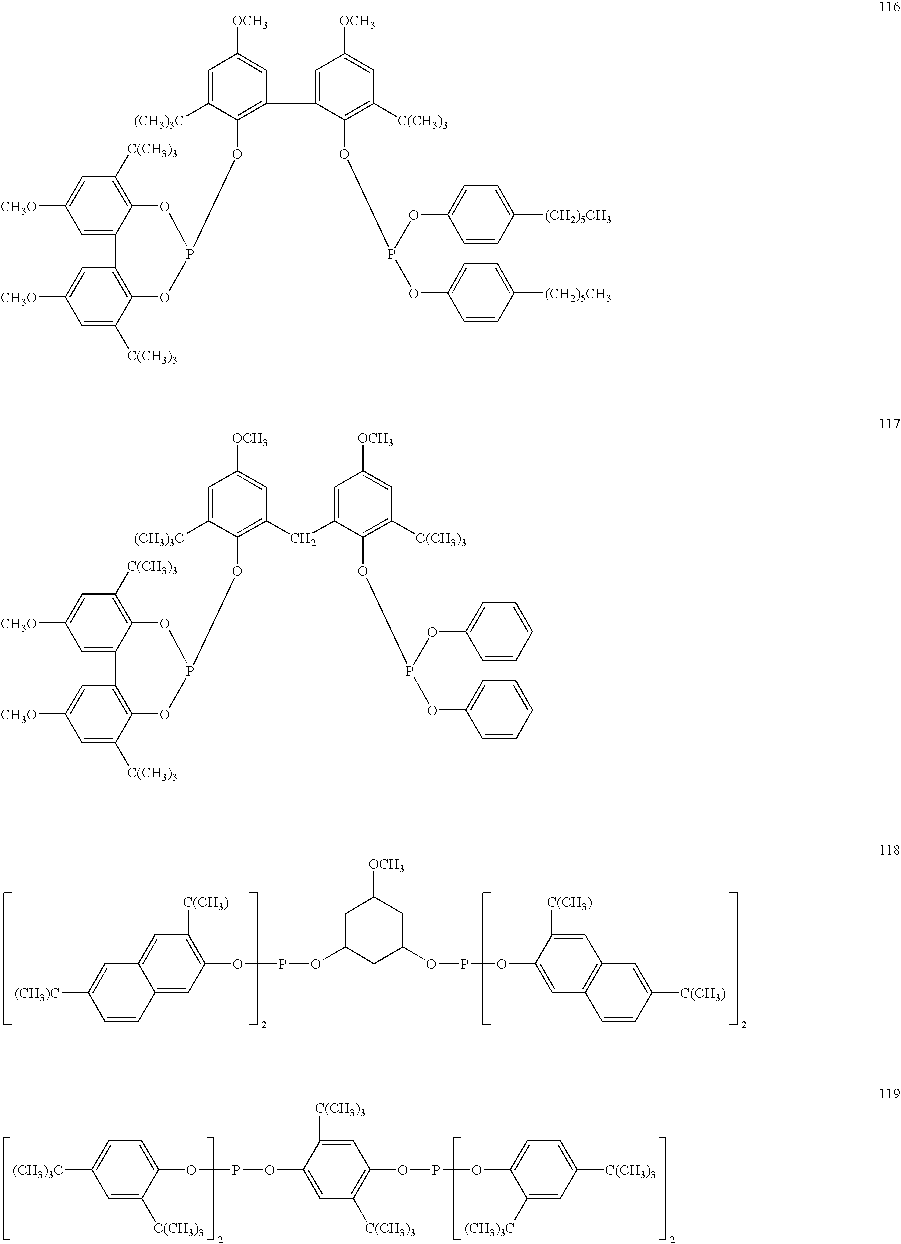 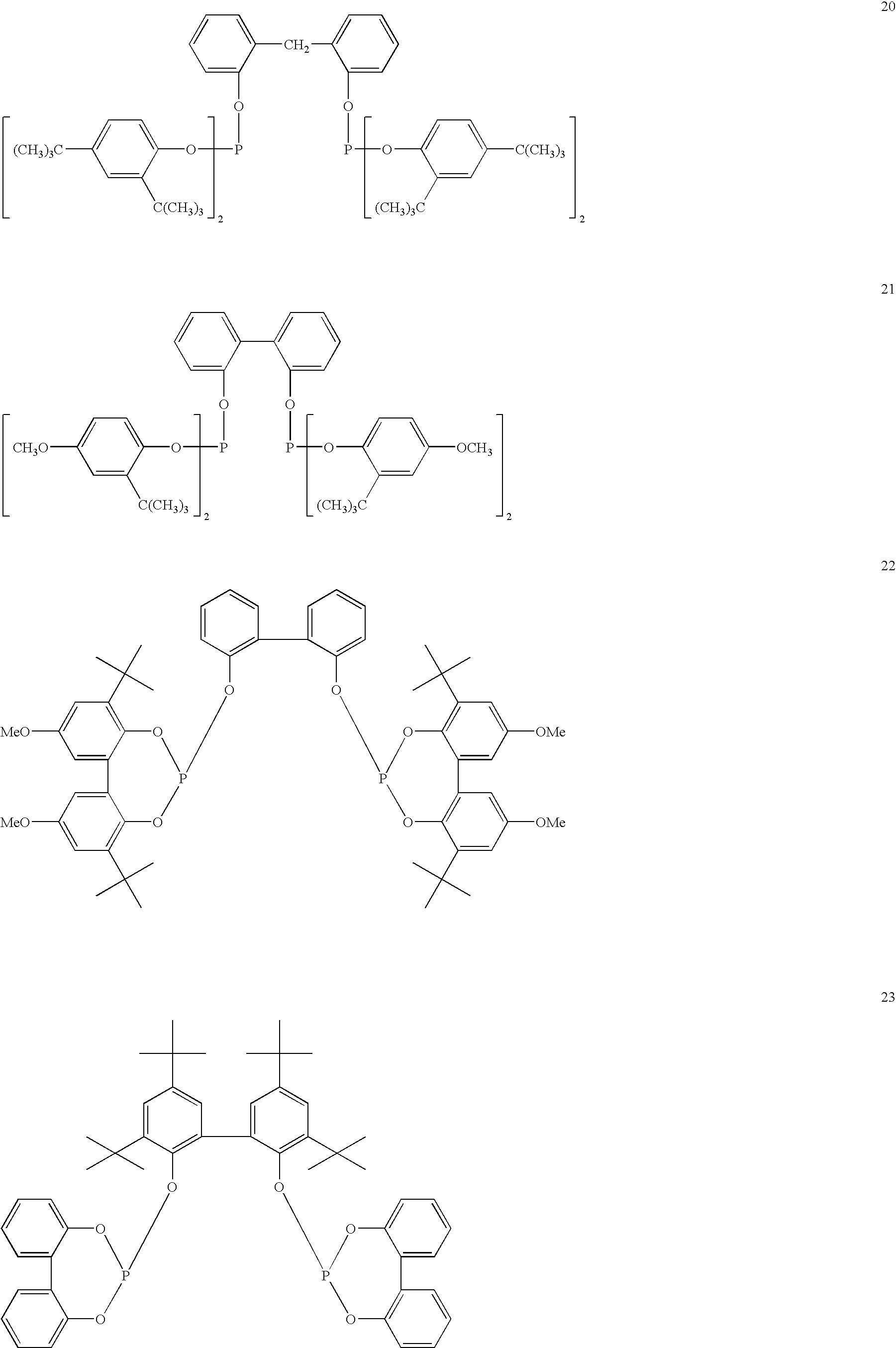 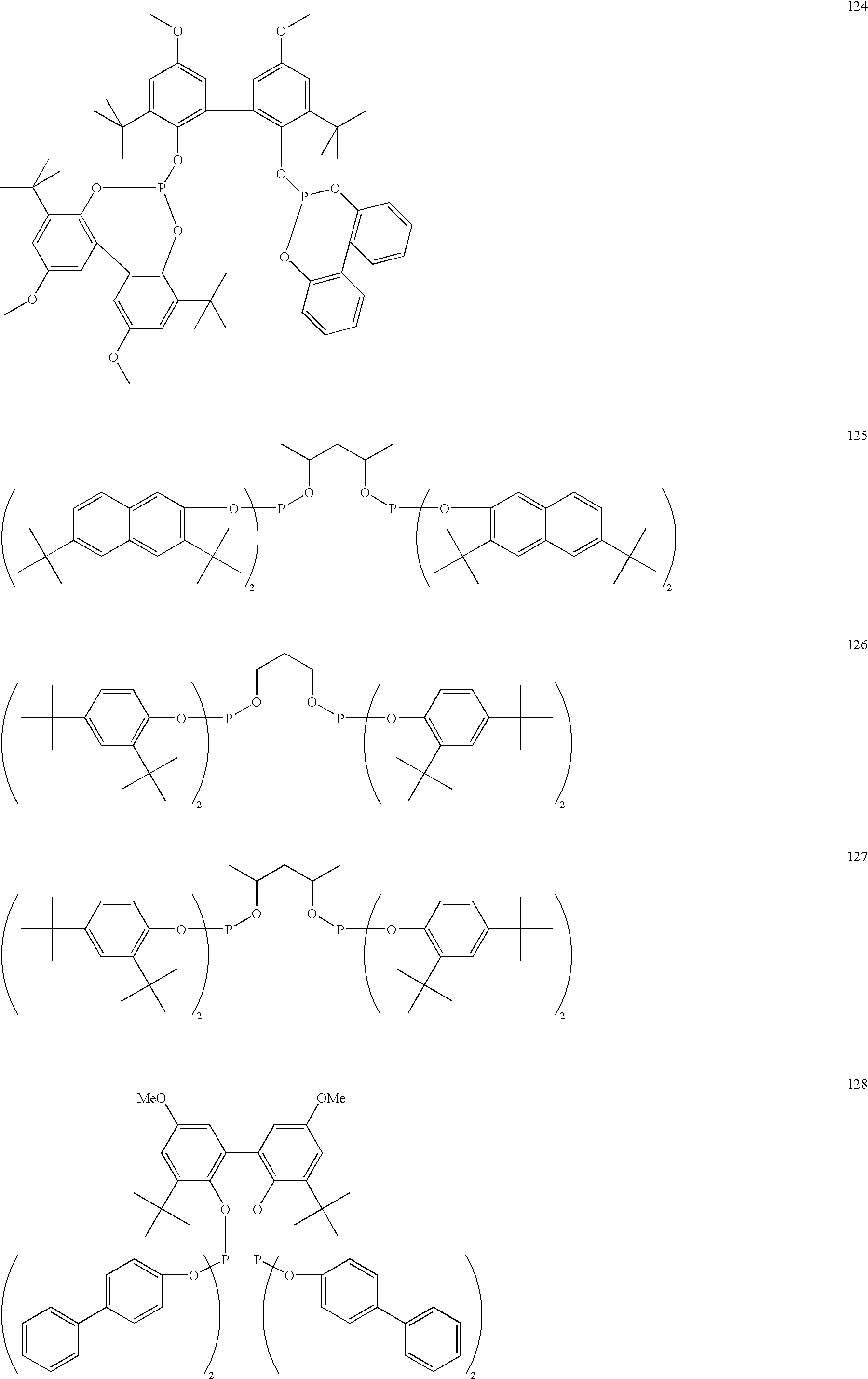 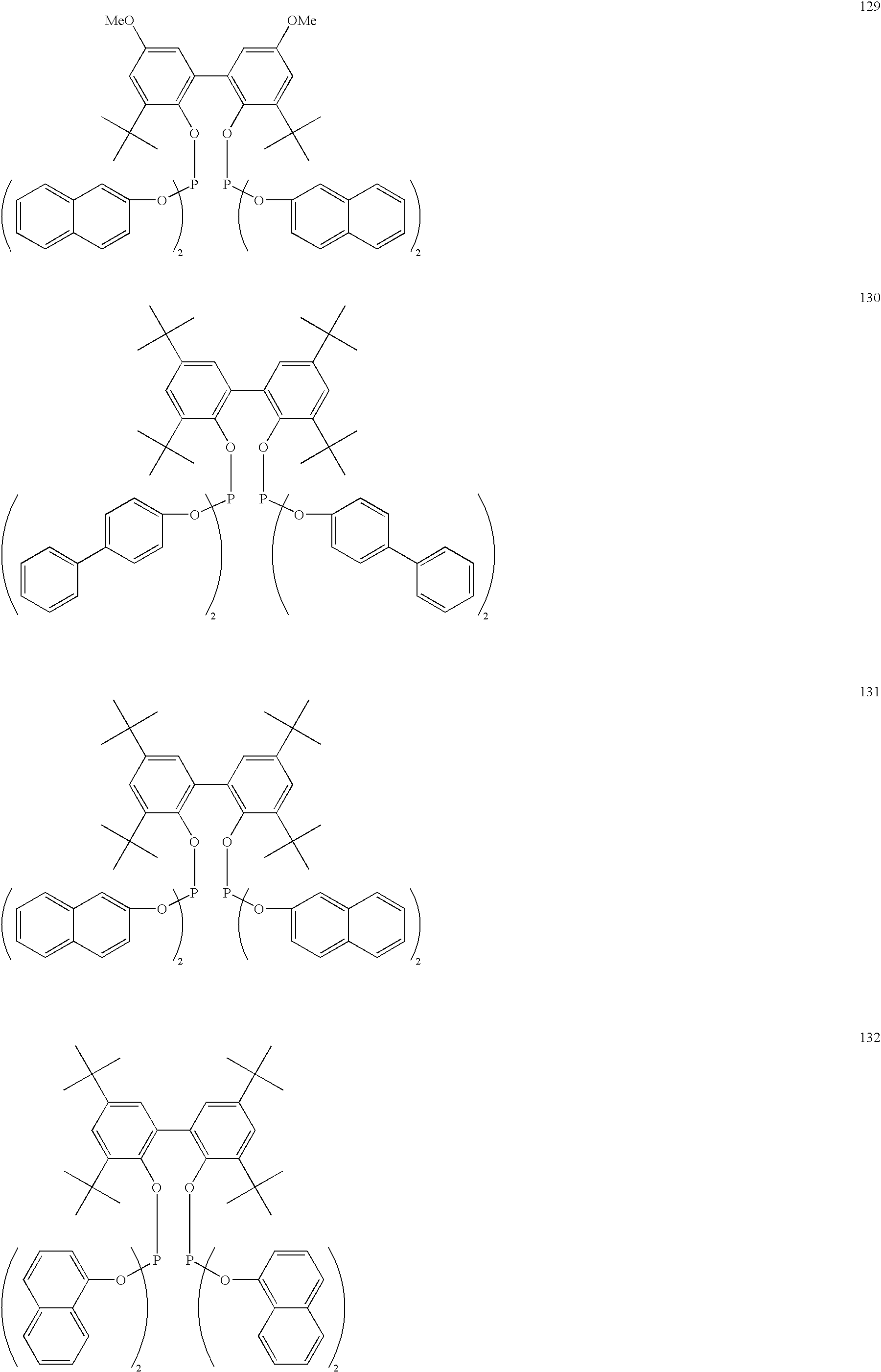 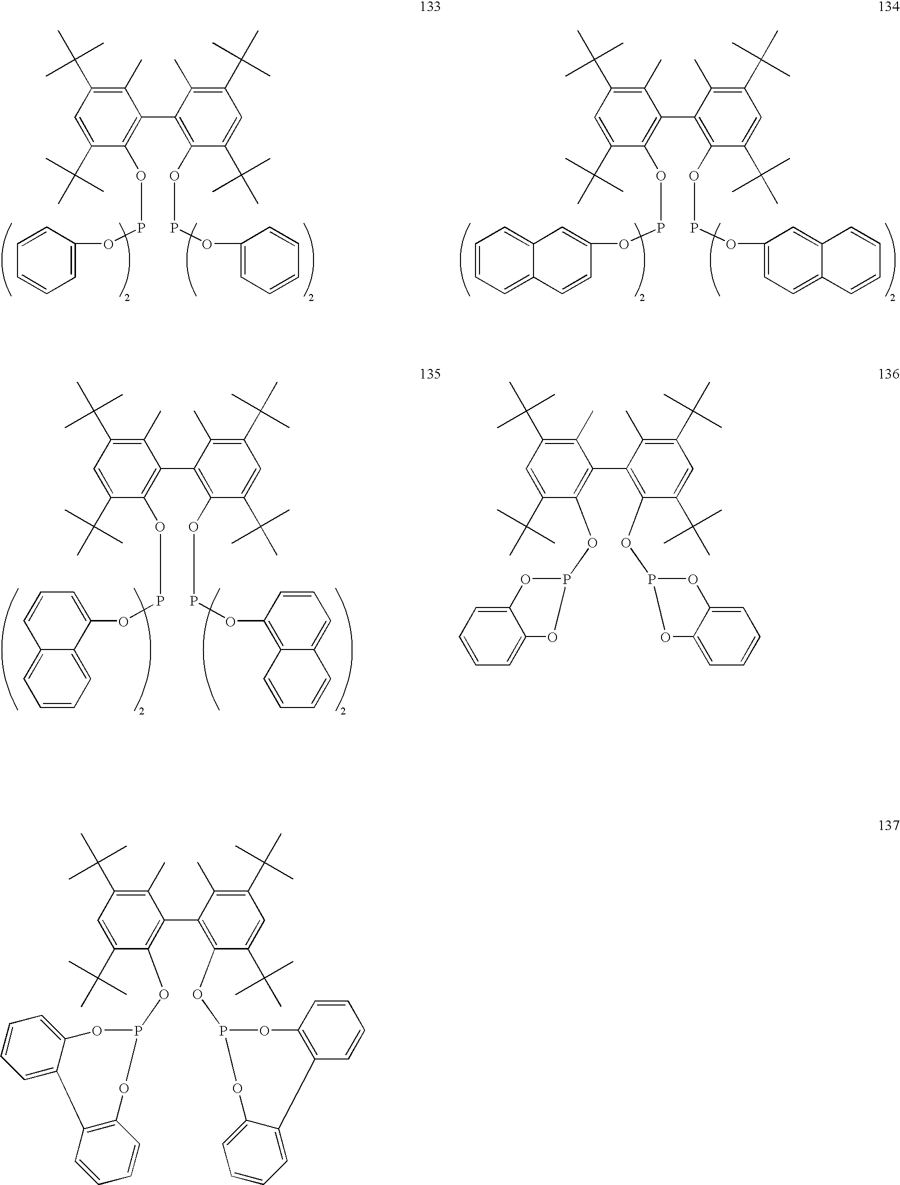
Such and other bisphosphite chelating ligands are subject matter of EP-A 213 369 and U.S. Pat. No. 4,769,498, and their preparation is described there.

Instead of the bisphosphite chelating ligands mentioned above, it is also possible to use monodentate monophosphite ligands of the general formula V
P(OR^{S}) (OR^{T}) (OR^{U}) V
for complexing the rhodium-hydroformylation catalyst and as free ligand in the process of the invention. The suitability of such ligands and their complexes with rhodium as catalysts for hydroformylation is known. In the monophosphite ligands of the general formula V, the radicals R^{S}, R^{T} and R^{U} are, independently of one another, identical or different organic groups which generally have from 1 to 30, preferably from 5 to 30, carbon atoms, for example substituted or unsubstituted alkyl, aryl, arylalkyl, cycloalkyl and/or heteroaryl groups. Owing to their increased stability toward hydrolysis and degradation, preference is here given to, in particular, sterically hindered monophosphite ligands as are described, for example, in EP-A 155 508. Merely for the purposes of illustration, the following monophosphite ligand structures may be mentioned by way of example:

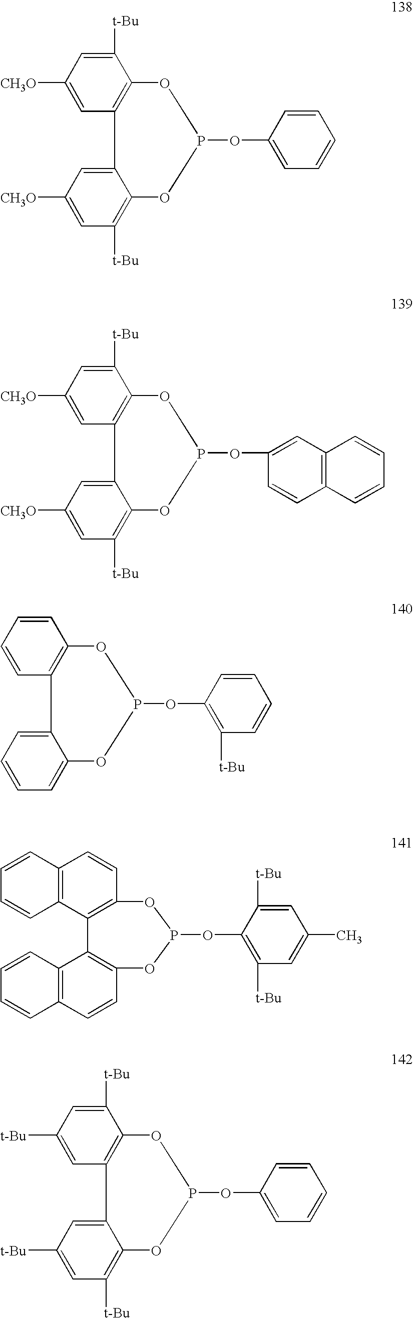

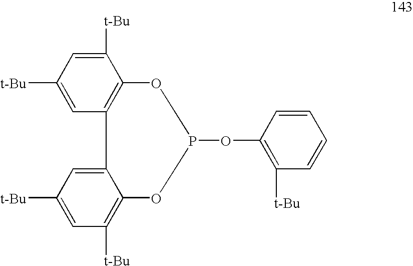

Known ligands for hydroformylation using rhodium complexes as catalysts also include bidentate ligands which have not only a phosphite group but also a phosphinite or phosphine group in the ligand molecule. Such ligands are described, inter alia, in WO 99/50214. Merely for the purposes of illustration, some ligands of this type are shown by way of example below:

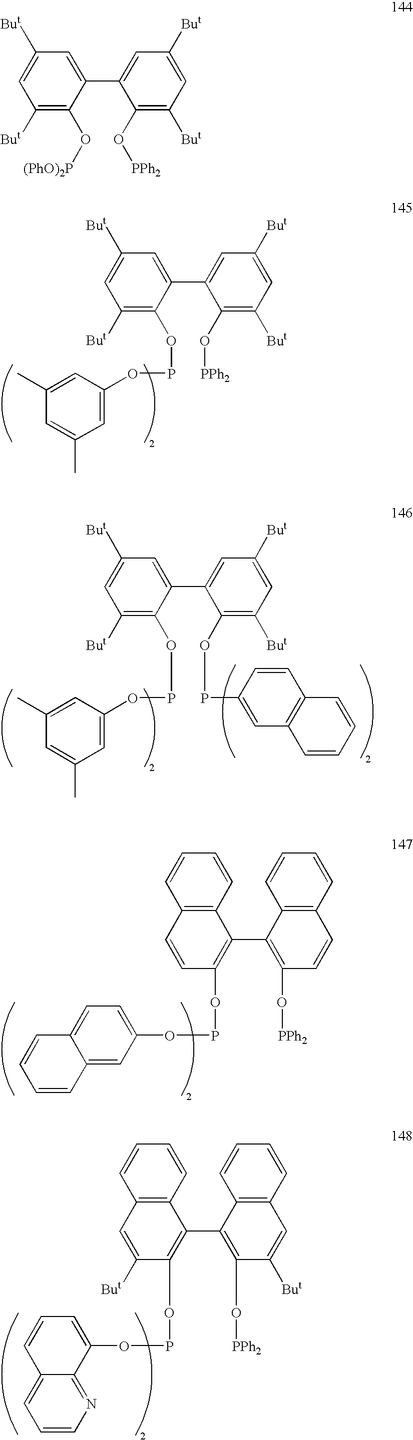

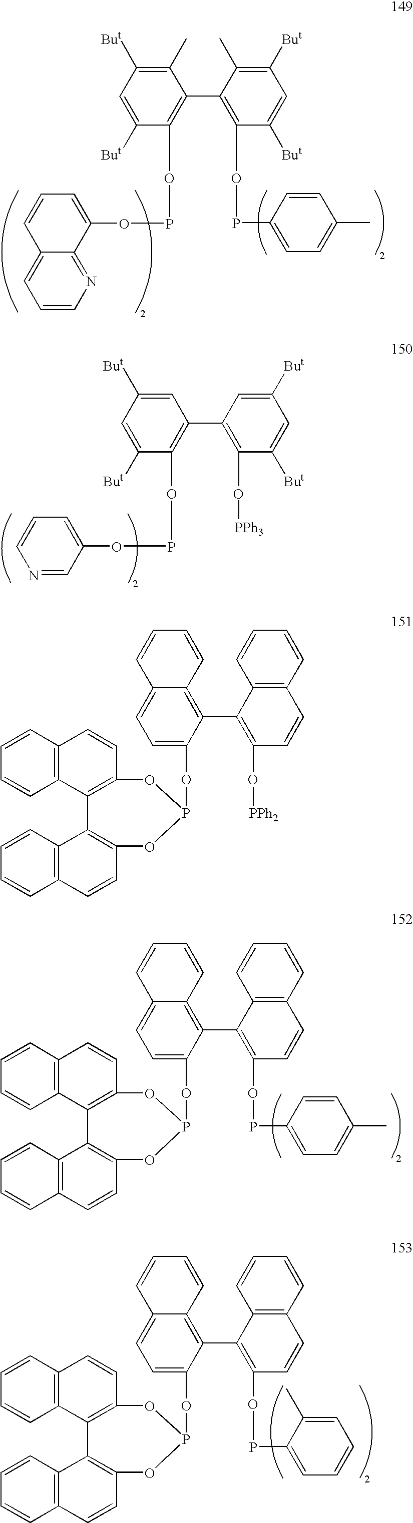

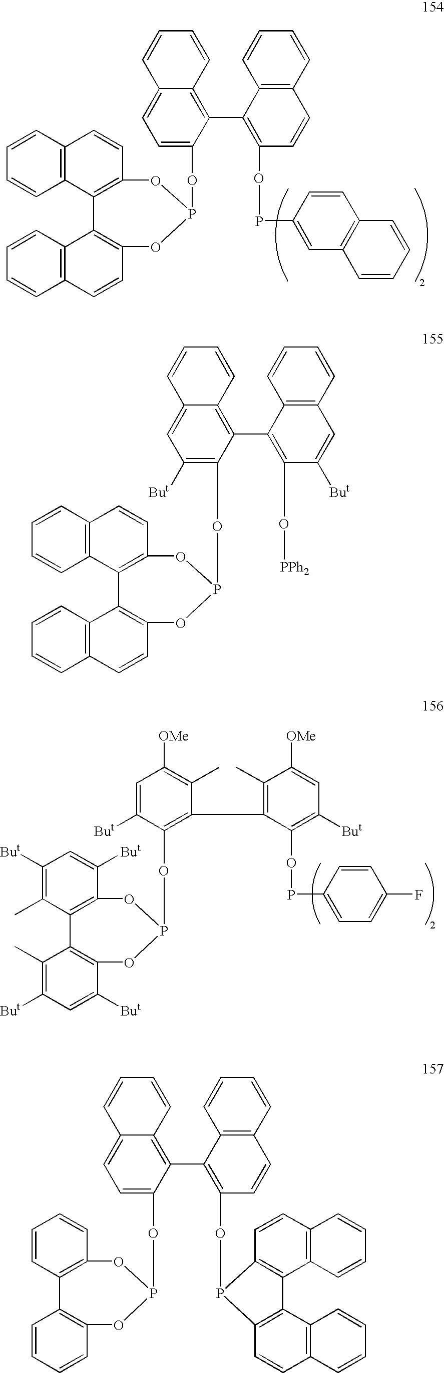

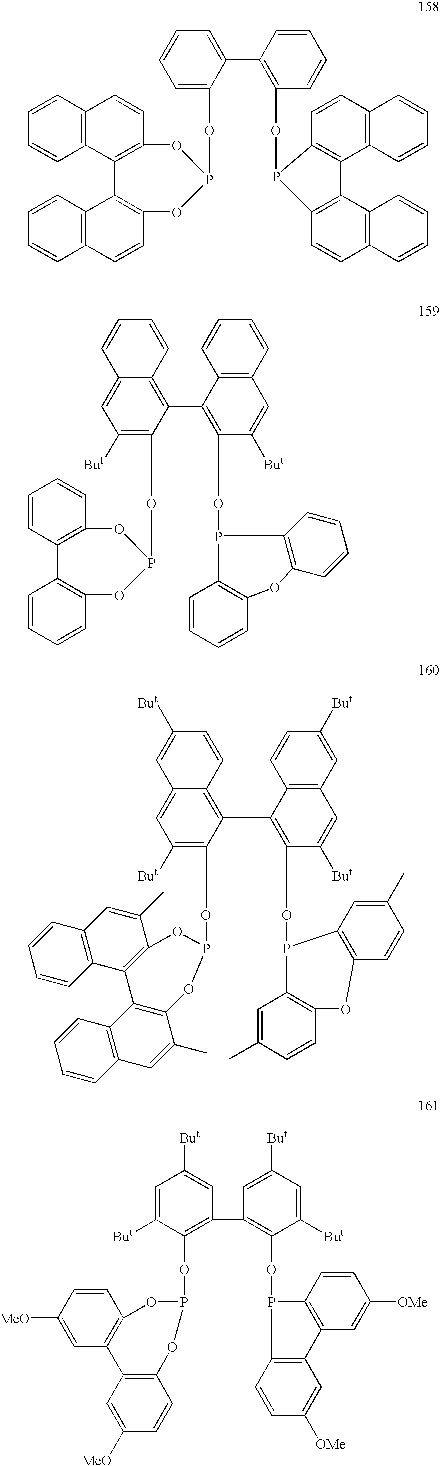

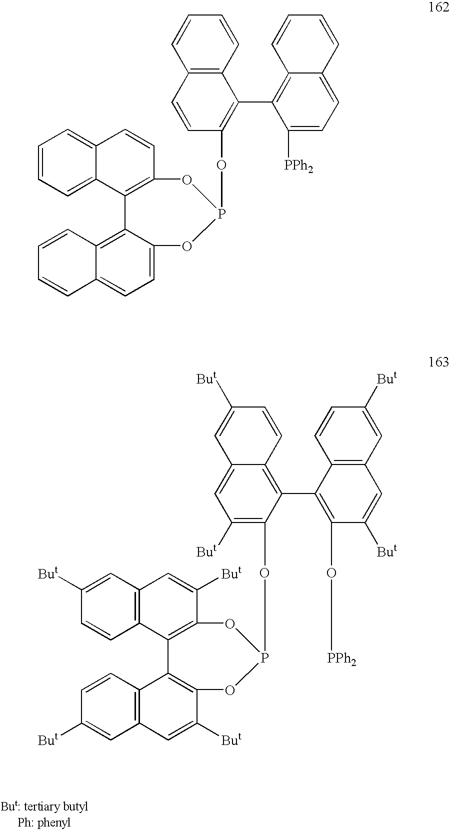

In general, catalytically active species of the general formula H_{g}Z_{d}(CO)ₑG_{f}, where Z is a metal of transition group VII, G is a phosphorus-, arsenic- or antimony-comprising ligand, for example one of the phosphorus-comprising ligands described above, and d, e, f, g are natural numbers which depend on the valence and type of the metal and the number of coordination sites occupied by the ligand G, are formed under hydroformylation conditions from the catalysts or catalyst precursors used in each case. e and f are preferably, independently of one another, at least 1, e.g. 1, 2 or 3. The sum of e and f is preferably from 2 to 5. The complexes of the metal Z with the ligands G used according to the invention can, if desired, additionally comprise at least one further ligand other than those used according to the invention, e.g. from the class of triarylphosphines, in particular triphenylphosphine, triaryl phosphites, triaryl phosphinites, triaryl phosphonites, phosphabenzenes, trialkylphosphines and phosphametallocenes. Such complexes of the metal Z with ligands used according to the invention and ligands other than those used according to the invention are formed, for example, in an equilibrium reaction after addition of a ligand to a complex of the general formula H_{g}Zₐ(CO)ₑG_{f}.

In a preferred embodiment, the hydroformylation catalysts are prepared in situ in the reactor used for the hydroformylation reaction. However, the catalysts of the process of the invention can, if desired, also be prepared separately and be isolated by customary methods. For the in-situ preparation of the catalysts, it is possible to react at least one compound of the general formulae I to V, a compound or a complex of a metal of transition group VIII, if desired one or more further additional ligands and, if appropriate, an activating agent in an inert solvent under the hydroformylation conditions.

Suitable rhodium compounds or complexes are, for example, rhodium(II) and rhodium(III) salts, e.g. rhodium(III) chloride, rhodium(III) nitrate, rhodium(II) sulfate, potassium rhodium sulfate, rhodium(II) or rhodium(III) carboxylate, rhodium(II) and rhodium(III) acetate, rhodium(III) oxide, salts of rhodic(III) acid, trisammonium hexachlororhodate(III), etc. Also suitable are rhodium complexes such as biscarbonylrhodium acetylacetonate, acetylacetonatobisethylenerhodium(I), etc. Preference is given to using biscarbonylrhodium acetylacetonate or rhodium acetate.

Ruthenium salts or compounds are likewise suitable. Suitable ruthenium salts are, for example, ruthenium(III) chloride, ruthenium(IV), ruthenium(VI) or ruthenium(VIII) oxide, alkali metal salts of ruthenium oxo acids, e.g. K₂RuO₄ or KRuO₄ or complexes such as RuHCl(CO)(PPh₃)₃. It is also possible to use the metal carbonyls of ruthenium, e.g. dodecacarbonyltrisruthenium or octadecacarbonylhexaruthenium, or mixed forms in which CO has been partly replaced by ligands of the formula PR₃, e.g. Ru(CO)₃(PPh₃)₂, in the process of the invention.

Suitable cobalt compounds are, for example, cobalt(II) chloride, cobalt(II) sulfate, cobalt(II) carbonate, cobalt(II) nitrate, their amine or hydrate complexes, cobalt carboxylates such as cobalt acetate, cobalt ethylhexanoate and cobalt naphthenoate. Here too, the carbonyl complexes of cobalt, e.g. octacarbonyl dicobalt, decacarbonyl tetracobalt and hexadecacarbonyl hexacobalt, can be used.

The abovementioned and further suitable compounds of cobalt, rhodium, ruthenium and iridium are known, are commercially available or their preparation is adequately described in the literature or they can be prepared by a person skilled in the art using methods analogous to those for the known compounds.

Suitable metals of transition group VIII are, in particular, cobalt and rhodium, and particular preference is given to rhodium.

As solvents, preference is given to using the aldehydes which are formed in the hydroformylation of the respective olefins, and also their higher-boiling subsequent reaction products, e.g. the products of the aldol condensation. Solvents which are likewise suitable are aromatics such as toluene and xylenes, hydrocarbons or mixtures of hydrocarbons, including for dilution of the abovementioned aldehydes and the subsequent products of the aldehydes. Further solvents are esters of aliphatic carboxylic acids with alkanols, for example ethyl acetate or Texanol®, ethers such as tert-butyl methyl ether and tetrahydrofuran. In the case of sufficiently hydrophilized ligands, it is also possible to use alcohols such as methanol, ethanol, n-propanol, isopropanol, n-butanol, isobutanol, ketones such as acetone and methyl ethyl ketone, etc. It is also possible to use “ionic liquids” as solvents. These are liquid salts, for example N,N′-dialkylimidazolium salts such as N-butyl-N′-methylimidazolium salts, tetraalkylammonium salts such as tetra-n-butylammonium salts, N-alkylpyridinium salts such as n-butylpyridinium salts, tetraalkylphosphonium salts such as trishexyl(tetradecyl)phosphonium salts, e.g. the tetrafluoroborates, acetates, tetrachloroaluminates, hexafluorophosphates, chlorides and tosylates.

Furthermore, the reactions can also be carried out in water or aqueous solvents comprising water together with a water-miscible solvent, for example an alcohol such as methanol, ethanol, n-propanol, isopropanol, n-butanol, isobutanol, a ketone such as acetone or methyl ethyl ketone or another solvent. For this purpose, use is made of ligands of the formula I or II which are modified with polar groups, for example ionic groups such as SO₃M, CO₂M where M=Na, K or NH₄, or such as N(CH₃)₄₊. The reactions then occur as a two-phase catalysis, with the catalyst being present in the aqueous phase and starting materials and products forming the organic phase. The reaction in the “ionic liquids” can also be carried out as a two-phase catalysis.

Preference is given to a process in which the hydroformylation catalyst is prepared in situ by reaction of at least one of the above-described ligands, a compound or a complex of a metal of transition group VIII and, if appropriate, an activating agent in an inert solvent under the hydroformylation conditions. However, the ligand-metal complexes can, if desired, also be prepared separately and be isolated by customary methods.

The hydroformylation reaction can be carried out continuously, semicontinuously or batchwise.

Suitable reactors for the continuous reaction are known to those skilled in the art and are described, for example, in Ullmanns Encyklopädie der technischen Chemie, Vol. 1, 3rd edition, 1951, p. 743 ff.

Suitable pressure-rated reactors are likewise known to those skilled in the art and are described, for example, in Ullmanns Encyklopädie der technischen Chemie, Vol.1, 3rd edition, 1951, p. 769 ff. In general, an autoclave which may, if desired, be provided with a stirrer and an internal lining is used when the process of the invention is carried out continuously.

The composition of the synthesis gas comprising carbon monoxide and hydrogen used in the process of the invention can be varied within a wide range. The molar ratio of carbon monoxide and hydrogen is generally from about 5:95 to 70:30, preferably from about 40:60 to 60:40.

The temperature in the hydroformylation reaction is generally in the range from about 20 to 180° C., preferably from about 40 to 140° C., in particular from about 50 to 120° C. The reaction is generally carried out at the partial pressure of the reaction gas at the reaction temperature selected. In general, the pressure is in the range from about 1 to 700 bar, preferably from 1 to 600 bar, in particular from 1 to 300 bar. The reaction pressure can be varied depending on the activity of the hydroformylation catalyst used. In general, the catalysts based on phosphorus-, arsenic- or antimony-comprising chelating pnicogen compounds allow reaction at relatively low pressures, for instance in the range from 1 to 100 bar, preferably from 5 to 50 bar.

The molar ratio of the ligand or ligands selected to the metal of transition group VIII in the hydroformylation medium is generally in the range from about 1:1 to 1000:1, preferably from 1:1 to 100:1, in particular from 1:1 to 50:1 and very particularly preferablyfrom 1:1 to 20:1.

The molar ratio of metal of transition group VII to substrate is usually less than 1 mol%, preferably less than 0.5 mol% and in particular less than 0.1 mol% and very particularly preferably less than 0.05 mol%.

The hydroformylation catalysts can be separated off from the output from the hydrofornylation reaction by customary methods known to those skilled in the art and can generally be reused for the hydroformylation.

The above-described catalysts can also be immobilized on a suitable support, e.g. of glass, silica gel, synthetic resins, etc., in an appropriate manner, e.g. by bonding via functional groups suitable as anchor groups or by adsorption, grafting, etc. They are then also suitable for use as solid-phase catalysts.

One embodiment of the present invention relates to the preparation of dialdehydes. In a preferred embodiment, the preparation of the dialdehydes is carried out batchwise.

Batch hydroformylation processes are known in principle to those skilled in the art. After the reaction is complete, the reactor is in general firstly depressurized. The synthesis gas liberated and any unreacted, unsaturated compounds can, if appropriate after work-up, be wholly or partly reused. The remaining contents of the reactor consist essentially of dialdehyde, high-boiling by-products (hereinafter also referred to as high boilers) and catalyst. To work up the contents of the reactor, the contents can be subjected to a single-stage or multistage fractionation, with at least one fraction enriched in dialdehyde being obtained. The fractionation to give a fraction enriched in dialdehyde can be carried out in various ways, for example by distillation, crystallization or membrane filtration, preferably by distillation. In a particularly preferred embodiment of the batch process, a reactor having a superposed distillation column is used, so that the distillation can be effected directly from the reactor. The distillation column is, if appropriate, provided with rectification trays in order to achieve a very good separation performance. The distillation can be carried out at atmospheric pressure or under reduced pressure. The fraction enriched in dialdehyde can be isolated at the top or in the upper region of the column, with at least one fraction depleted in dialdehyde being able to be isolated at the bottom or in the lower region of the column. Suitable columns, temperature parameters and pressure parameters are known to those skilled in the art. The fraction enriched in dialdehyde can, if appropriate, be subjected to a further purification step. The fraction depleted in dialdehyde comprises essentially high boilers and the catalyst. The catalyst can be separated off by customary methods known to those skilled in the art and can generally, if appropriate after work-up, be reused in a further hydroformylation.

In a further preferred embodiment, the preparation of the dialdehydes is carried out continuously. In the continuous process, an unsaturated compound is subjected to hydroformylation in one or more reaction zones. An output is taken from the reaction zone and this is in general firstly depressurized. Here, unreacted synthesis gas and unsaturated compounds are liberated and these are generally, if appropriate after work-up, recirculated to the reaction zone. The fractionation of the remaining output to give a fraction enriched in dialdehyde can be carried out by means of customary measures known from the prior art, for example by distillation, crystallization or membrane filtration. Suitable distillation plants are known to those skilled in the art. Thin film evaporators are also suitable. In the fractional distillation, a fraction consisting essentially of high boilers and catalyst is taken off from the bottom or the lower region of the column and this can be recirculated directly to the reaction zone. However, all or part of the high boilers is preferably discharged prior to recirculation and the catalyst is recirculated, if appropriate after work-up, to the reaction zone. At least one fraction which is enriched in dialdehyde and may also comprise unsaturated monoaldehyde is taken off at the top or in the upper region of the column. The fraction which is enriched in dialdehyde and further comprises unsaturated monoaldehyde is advantageously subjected to at least one further fractionation to give at least one fraction enriched in unsaturated monoaldehyde and a fraction enriched in dialdehyde. The phase which is enriched in unsaturated monoaldehyde is recirculated to the reaction zone and the phase which is enriched in dialdehyde is worked up.

In a preferred embodiment, diolefin comprising internal double bonds or mixtures of diolefins which comprise diolefin having internal double bonds can be reacted. The reaction can, for example, be carried out as follows:

In the case of diolefins which comprise both terminal and internal double bonds, it is advantageous to carry out the reaction under conditions under which the terminal double bonds are firstly preferentially hydroformylated with high n selectivity and the internal double bonds are then reacted under isomerizing hydroformylation conditions to give aldehydes having a high proportion of n-product. In this way, mixtures of, for example, 1,7-octadiene with at least one further diolefin having in each case at least one further internal double bond can be successfully hydroformylated to give the corresponding terminal dialdehyde. Mention may be made by way of example of the reaction of 1,7-octadiene contaminated with 1,6-octadiene, as can be obtained, for example, in the above-described metathesis reaction according to the invention, when appropriate conditions are selected. Such a hydroformylation process is described in the unpublished German patent application number 10349482.0, which is fully incorporated by reference. The olefin composition is preferably reacted at a total pressure of from 10 to 40 bar with synthesis gas having a carbon monoxide:hydrogen molar ratio of from 4:1 to 1:2 to an extent of from 40 to 95% based on olefins having terminal double bonds in the first reaction zone and the output from the hydroformylation is reacted at a total pressure or from 5 to 30 bar with synthesis gas having a carbon monoxide:hydrogen molar ratio of from 1:1 to 1:1000 in one or more downstream reaction zones, with the total pressure in one or more downstream reaction zones preferably being in each case lower than that in the preceding reaction zone.

This can be achieved similarly in batch operation by changing the reaction conditions after the desired conversion of the terminal double bonds.

The present invention further relates to the use of 1,10-decanedial prepared by the above-described route for preparing optionally olefinically unsaturated 2,15-hexadecanedione and for preparing 3-methylcyclopentadecanone (muscone) and/or its partially hydrogenated analogs by intramolecular aldol reaction of optionally olefinically unsaturated 2,15-hexadecanedione and, if appropriate, subsequent hydrogenation.

It is known that it is possible to use 1,7-octadiene which has been prepared in another way for hydroformylation and synthesis of muscone, for example 1,7-octadiene which has been prepared directly from butadiene by dimerization or by a route via octadienols and their derivatives and also via cyclooctene by pyrolysis. However, these starting materials which have not been prepared according to the invention have to be subjected to complicated purification in order to remove impurities such as conjugated dienes and oxygen-comprising impurities. 2,15-Hexadecanedione and its olefinically unsaturated analogs are important intermediates for the synthesis of macrocyclic ketones, in particular for the synthesis of 3-methylcyclopentadecanone (muscone) of the formula VI, one of the most important musk fragrances.

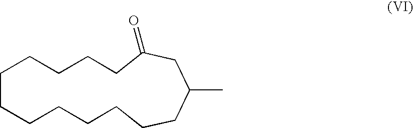

A good synthetic route to 2,15-hexadecanedione or its olefinically unsaturated analogs is accordingly one of the prerequisites for an economically satisfactory synthesis of muscone or its analogs which can be carried out on an industrial scale.

DE-A 39 18 015 describes a process for preparing muscone and open-chain and sometimes olefinically unsaturated 2,15-diketones as intermediates for this process and also their preparation. Two reaction sequences are disclosed as methods of preparing the optionally olefinically unsaturated 2,15-hexadecanediones: one method is to dehydrogenate 1,10-decanediol oxidatively to form 1,10-decanedial and then react this with a suitable Wittig reagent. As an alternative, 1,6-hexanediol can likewise be dehydrogenated oxidatively to form the corresponding dialdehyde and this can subsequently be reacted with 2 equivalents of a vinyl Grignard reagent to give 1,9-decadiene-3,8-diol. The desired intermediate is obtained therefrom by a Caroll reaction with an alkyl acetoacetate.

The optionally olefinically unsaturated 2,15-hexadecanediones are then cyclized by intramolecular aldol condensation in the gas phase and subsequently hydrogenated catalytically to give muscone.

Further routes to optionally unsaturated 2,15-hexadecanediones and to equivalent intermediates and to muscone may be found in the same document.

JP-A 2000001452 describes a process for preparing diketones, including 2,15-hexadecanedione, from dialdehydes by base-catalyzed aldol reaction with acetone under hydrogenating conditions.

In addition, M. Baumann et al. in Tetrahedron Lett. 1976, 3585, describe the preparation of muscone from cyclododecenone by Nazarov cyclization with 3-butynol.

A synthesis of muscone from tetradecanedioic acid by methylation of the acid chloride to form 2,15-hexadecadione and subsequent aldol cyclization and hydrogenation is described by S. Ellwood and T. Haines in Current Topics in Flavours and Fragrances, 1999, Kluwer Academic Publishers, Amsterdam, 79-95.

In J. Chem. Soc., Chem. Comm. 1972, 802, R. Baker et al. teach the preparation of muscone by nickel-catalyzed coupling of butadiene, allene and carbon monoxide and subsequent hydrogenation.

In Seifen-Öle-Fette-Wachse, 1989, 115, 538-545, S. Warwel et al. describe the preparation of 8-hexadecene-2,15-dione from cycloheptene and subsequent reaction to form muscone by aldol condensation and subsequent hydrogenation.

A further object of the present invention was accordingly to provide an alternative process by means of which 2,15-hexadecanedione or its olefinically unsaturated analogs can be prepared in an economical way which can readily be carried out on an industrial scale.

The 1,1 0-decanedial obtained according to the invention is particularly suitable for the preparation of 2,15-hexadecanedione of the formula VII and 3,13-hexadecadiene-2,15-dione of the formula VII, with the latter being able to be present in the form of trans/cis mixtures in respect of the configuration of.the C-C double bonds. In this process, decanedial is preferably reacted with acetone in the presence of a base and, if appropriate, catalytically hydrogenated or decanedial is reacted with ethyl acetoacetate in the presence of a base and the product is subsequently hydrolyzed and decarboxylated and, if appropriate, finally catalytically hydrogenated.

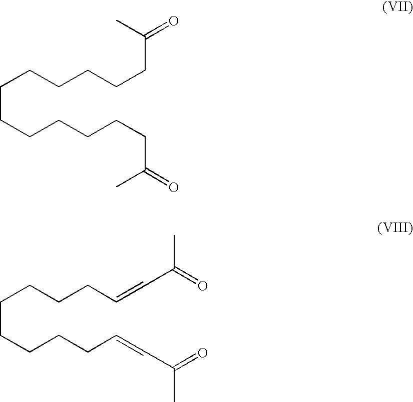

The ease of preparation of these intermediates also makes the process suitable, in combination with known process steps, for preparing 3-methylcyclopentadecanone (muscone) of the formula VI. The incorporation of the process of the invention for preparing optionally olefinically unsaturated 2,15-hexadecanediones into a synthetic route to muscone or its olefinically unsaturated analogs thus represents a further aspect of the present invention.

The 1,0-decanedial serving as starting compound can be obtained in various ways. For example, the dialdehyde can be obtained, as described in DE-A 39 18 015, by oxidative dehydration of 1,10-decanediol, or else, for example, by reduction of the 1,10-dicarboxylic acid. A preparative method which is preferred for the purposes of the process of the invention is the double hydroformylation of 1,7-octadiene or mixtures of 1,6- and 1,7-octadiene, preferably 1,7-octadiene, over rhodium catalysts carried out as described above. 1,7-Octadiene or the mixtures of 1,6- and 1,7-octadiene can preferably be prepared by metathesis of cyclohexene in the presence of ethylene.

To prepare 2,15-hexanedecadione or to convert it further into muscone, 1,10-decanedial is reacted either with acetone or with ethyl acetoacetate in the presence of a base and, if appropriate, the product is subsequently hydrogenated catalytically.

The reaction with acetone is carried out in the presence of a suitable base which catalyzes the aldol condensation, e.g. NaOH, KOH, LiOH, Ba(OH)₂, Ca(OH)₂, CsOH, RbOH or amines such as diazabicyclo-1,5-[5.4.0]undecane (DBU), piperidine or triethylamine or else basic aluminum oxide (Al₂O₃). The reaction can be carried out continuously, semicontinuously or batchwise in a homogeneous or heterogeneous phase. In general, from about 2 to about 30 mol, preferably from about 6 to about 14 mol of acetone and from about 2 to about 60 mol, preferably from about 6 to about 30 mol, of the catalytically active base are used per mole of 1,10-decanedial to be reacted.

The reaction can be carried out under conditions known per se to those skilled in the art. Thus, for example, acetone or a solvent which is inert under the reaction conditions, e.g. toluene, diethyl ether or tetrahydrofuran, serves as solvent. The chosen base is usually initially charged together with the solvent and the dialdehyde is added thereto. Better selectivities are generally achieved when addition is carried out stepwise or continuously. At temperatures of about 100° C., the reaction is generally concluded after a few hours.

The reaction products obtained in this way can subsequently be purified by methods known per se to those skilled in the art, e.g. crystallization, chromatography or distillation. The olefinically unsaturated reaction products can subsequently be catalytically hydrogenated, likewise in a known manner. Preferred catalysts for this are catalysts which are able to hydrogenate olefinic double bonds preferentially in the presence of carbonyl groups. Examples are, inter alia, palladium-comprising catalysts.

In a preferred embodiment, the aldol condensation of 1,10-decanedial with acetone is carried out under hydrogenating conditions, i.e. in a single-stage process. For this purpose, the reactants are reacted under a hydrogen atmosphere in the presence of a hydrogenation-active catalyst. Suitable catalysts are, for example, ones in which the hydrogenation-active component or components has/have been applied to a support such as Al₂O₃, TiO₂ or ZrO₂, preferably Al₂O₃. Suitable hydrogenation-active components are transition metals such as Ru, Rh, Ir, Pt, Co and Pd, particularly preferably Pd. The hydrogenation-active components mentioned may, if appropriate, comprise further metals, preferably lanthanides or compounds thereof. Among these, particular preference is given to the lanthanides Pr, Nd, Eu, Gd, Dy, Ho, Er and Yb. Very particular preference is given to using a catalyst comprising Pr-doped Pd as hydrogenation-active component applied to Al₂O₃ as support.

The aldol condensation under hydrogenating conditions can be carried out batchwise, semicontinuously or fully continuously. Depending on the mode of operation, the reaction can be carried out in suitable reactors, e.g. in stirred vessels, tube reactors, flow reactors, loop reactors or cascades of stirred vessels. The reaction is usually carried out at temperatures of from about 10 to about 280° C. under hydrogen pressures of from about 1 to about 100 bar. The reaction is then generally concluded after a few hours. The conversion is often quantitative after about 2 hours.

To prepare the olefinically unsaturated compounds of the formula VII, which are obtained in the form of trans/cis mixtures in respect of the olefinic double bonds, the aldol reaction of decanedial with acetone is carried out under conventional, i.e. nonhydrogenating, conditions. Basic catalysts without hydrogenation-active component or components are suitable for this purpose. In addition, the hydrogen atmosphere is dispensed with.

The compounds of the formulae VII and VIII which can be obtained in this way are likewise suitable for cyclization by intramolecular aldol condensation as is described comprehensively in, for example, DE-A 39 18 015.

The procedure described enables muscone, which is valued as a fragrance, its partially hydrogenated analogs and in principle also many further macrocyclic ketones by a simple and economically advantageous route which can readily be carried out on an industrial scale.

If the final hydrogenation of the primary aldol condensation product is carried out under asymmetric conditions, e.g. using a chiral nonracemic, enantioselective catalyst, it is possible to obtain muscone in optically active form.

The following examples serve to illustrate the present invention without restricting it in any way:

### EXAMPLES

### Example 1

### Preparation of 1,7-octadiene by Metathesis

40 g of a catalyst comprising 10% by weight of Re₂O₇ on Al₂O₃ (4 mm extrudates) were placed in an autoclave, the autoclave was pressurized with ethylene to a pressure of 40 bar and 180 ml of cyclohexene were introduced. The reaction mixture was heated to 40° C. and the ethylene pressure was increased to 200 bar. The reaction mixture was subsequently stirred for 24 hours. A conversion of 1.3% was obtained at a selectivity to 1,7-octadiene of 89%. The space-time yield based on the catalyst was 0.002 kg/kgh.

### Example 2a

### Continuous Preparation of 1,7-octadiene by Metathesis

40 g of a catalyst comprising 10% by weight of Re₂O₇ on Al₂O₃ (1.5 mm extrudates) were placed in a tube reactor. At 60° C., 60 g/h of cyclohexene and 80 g/h of ethylene were fed in continuously at a pressure of 80 bar. After 15 hours, a sample of the output from the reaction was analyzed by gas chromatography. At a conversion of 7.9%, 97.3% of 1,7-octadiene and 2.0% of 1,7,13-tetradecatriene were obtained. 122 g of 1,7-octadiene were isolated from the reaction product obtained after 30 hours by distillation. The space-time yield based on the catalyst was thus 0.1 kg/kgh.

### Example 2b

### Continuous Preparation of 1,7-octadiene by Metathesis With Increase of the Temperature and Recirculation of Starting Material

30 g of a catalyst comprising 10% by weight ot Re₂O₇ on Al₂O₃ (1.5 mm extrudates) mixed with 30 ml of molecular sieves 13X were placed in a tube reactor. At 25° C., 60 g/h of cyclohexene and 41 g/h of ethylene were fed in continuously at a pressure of 80 bar. For this purpose, the ethylene recovered from the product stream in a stripping column and the cyclohexene recovered in a distillation column were supplemented with fresh starting materials. After reaction at 25° C. for half an hour, the temperature in the reactor was increased continuously at a rate of 1.5° C./h to 80° C. The reaction was subsequently continued at this temperature. 98.3% of 1,7-octadiene was obtained at an average conversion of 8.0%. A total of 265 g of 1,7-octadiene were isolated over a period of 42 hours. The space-time yield based on the catalyst was thus 0.21 kg/kgh.

### Example 3

### Hydroformylation of 1,7-octadiene (85% pure) Using Rh/ligand A Synthesis of Ligand A

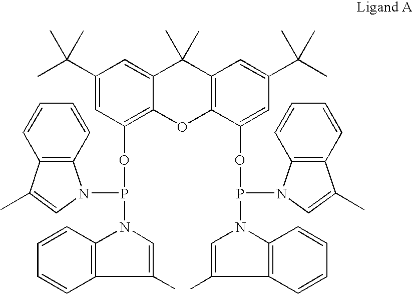

28.5 g (218 mmol) of 3-methylindole (skatole) together with about 50 ml of dry toluene were placed in a reaction vessel at room temperature and the solvent was distilled off under reduced pressure (removal of traces of water). This procedure was repeated once more. The residue was subsequently taken up in 700 ml of dry toluene under argon and the mixture was cooled to −65° C. At −65° C., 14.9 g (109 mmol) of PCl₃ were added first and 40 g (396 mmol) of triethylamine were then slowly added. The mixture was warmed to room temperature over a period of 16 hours and subsequently refluxed for 16 hours. 19.3 g (58 mmol) of 4,5-diydroxy-2,7-di-tert-butyl-9,9-dimethylxanthene in 300 ml of dry toluene were then added at room temperature and the mixture was refluxed for 16 hours. The triethylamine hydrochloride formed was filtered off and washed once with toluene. After evaporation of the organic phases, the residue was recrystallized twice from hot ethanol. Drying under reduced pressure gave 36.3 (71% of theory) of a colorless solid. ³¹P-NMR (298 K) d: 105 ppm.

5.1 mg of Rh(CO)₂acac (acac=acetylacetonate) and 187 mg of the ligand A were each dissolved in 5 g of toluene and introduced into a 100 ml steel autoclave which had been made inert by means of synthesis gas (CO:H₂=1:1) and was provided with a sparging stirrer. The autoclave was then pressurized with 10 bar of synthesis gas (CO:H₂=1:1) and the contents were treated with gas at 80°0 C. After 1 hour, the autoclave was depressurized. 10 g of 1,7-octadiene (85% pure, further comprising other olefins and diolefins, in particular 1,6-octadiene) were subsequently introduced by means of a syringe. The mixture was then hydroformylated at 80° C. for 6 hours (10 ppm of Rh; ligand A:Rh=10:1) and a sample was then analyzed by means of gas chromatography. 90% of dials having a linearity of 98% (linearity=1,10-decanedial/sum of dials) was obtained at a conversion of 99% (based on 1,7-octadiene).

### Example 4

### Preparation of Muscone

4.1 Hydroformylation of 1,7-octadiene

5 mg of Rh(CO)₂acac (acac=acetylacetonate) and 181 mg of the ligand A (prepared as described below) were each dissolved in 5 g of toluene. The two solutions were mixed and heated to 60° C. The reaction vessel was subsequently pressurized with 10 bar of a 1:1 mixture of CO₂ and H₂ (“synthesis gas”). After 30 minutes, the reaction vessel was depressurized, 10 g of 1,7-octadiene were added, the vessel was pressurized with 20 bar of synthesis gas and hydroformylation was carried out at 60° C. for 6 hours. Decanedial was obtained with a dialdehyde selectivity of 84% and a linearity of 98% (linearity=1,10-decanedial/sum of dials) at a conversion of 98%.
4.2 Aldol condensation of decanedial with acetone under hydrogenating conditions

10 g of decanedial and 40 g of acetone were admixed with 1.6 g of a catalyst comprising 0.5% by weight of Pd and 5% by weight of PrO₂ (in each case based on the finished catalyst) on Al₂O₃ (4 mm extrudates). The reaction vessel was subsequently pressurized with 10 bar of hydrogen, heated to 180° C. and a pressure of 40 bar was set. After 24 hours, the reaction was stopped. The crude product was analyzed by gas chromatography. 2.8% of dodecanone, 20.8% of tridecanal-12-one and 69.5% of 2,15-hexadecanedione together with 6.9% (in each case GC-% by area) of other by-products were obtained at quantitative conversion. When 45 g of acetone and 3.2 g of the catalyst were used under otherwise unchanged conditions, 2.6% of dodecanone, 9.3% of tridecanal-12-one and 73.8% of 2,15-hexadecanedione together with 14.3% (in each case GC-% by area) of other by-products were obtained, likewise at quantitative conversion.
4.3 Intramolecular aldol condensation of 2,15-hexadecanedione

A mixture of 3 g of 2,15-hexadecanedione, 10 ml of water and 60 ml of toluene was vaporized in a tube reactor. The mixture was passed in a stream of nitrogen (10 l/h) over a bed of a catalyst comprising 2% by weight of K₂O (based on the finished catalyst) on TiO₂ (4 mm extrudates) at 370° C. for a period of 2 hours. The output from the reactor was condensed and analyzed by gas chromatography. 86% (GC-% by area) of a mixture of dehydromuscone isomers was obtained at a conversion of 60%.
4.4 Hydrogenation of the dehydromuscone isomers

2 g of the mixture of dehydromuscone isomers obtained as described above together with 40 ml of cyclohexane were placed in a reaction vessel and 1 g of a catalyst comprising 10% by weight of Pd on carbon (Pd/C 10%) were added. The reaction vessel was pressurized with 10 bar of hydrogen and the contents were stirred at 70° C. for 1 hour. The crude product obtained was analyzed by gas chromatography. Muscone was obtained in a yield of 98.5% (GC-% by area) at quantitative conversion.

When a catalyst comprising 10% by weight of Pd on Al₂O₃ (Pd/Al₂O₃ 10%) was used, muscone was obtained in a yield of 93.1% (GC-% by area) at a conversion of 98%.

1. A process for preparing 1,7-octadiene by metathesis of cyclohexene with ethylene, wherein the unreacted starting materials and any relatively high-boiling by-products obtained are recirculated in purified form to the reaction mixture.

2. The process according to claim 1, wherein
a. ethylene and cyclohexene are brought into contact with a suitable catalyst in a reactor R,
b. the output from the reaction is transferred to a distillation apparatus D1 and the reaction product is separated into a relatively low-boiling fraction comprising the unreacted ethylene and a relatively high-boiling fraction,
c. the relatively high-boiling fraction obtained in this way is transferred to a further distillation apparatus D2 and separated into a relatively low-boiling fraction comprising the unreacted cyclohexane and a relatively high-boiling fraction comprising 1,7-octadiene and
d. the relatively low-boiling fractions separated off in the distillation apparatuses D1 and D2 are each wholly or partly recirculated to the reactor R.

3. The process according to claim 1, wherein the relatively high-boiling reaction product mixture from the distillation apparatus D2 is transferred to a further distillation apparatus D3 and 1,7-octadiene is separated from relatively high-boiling by-products.

4. The process according to claim 1, wherein the relatively high-boiling by-products separated off in distillation apparatus D3 are wholly or partly recirculated to the reactor R.

5. The process according to claim 1, carried out continuously.

6. The process according to claim 1, wherein cyclohexane which is essentially free of cyclohexanol and/or cyclohexanone is used.

7. The process according to claim 1, wherein the molar ratio of ethylene used to cyclohexane used is from 1 to 10.

8. The process according to claim 1, wherein the molar ratio of ethylene used to cyclohexane used is from 2 to 4.

9. The process according to claim 1, wherein a catalyst comprising Re, W, Mo, Ru, Os, Ta and/or Nb is used.

10. The process according to claim 1, wherein a catalyst comprising an Re-comprising compound applied to Al₂O₃ as support is used.

11. The process according to claim 1, wherein a catalyst comprising, based on the total weight of the finished catalyst, from 6 to 12% by weight of Re₂O₇ applied to Al₂O₃ as support is used.

12. The process according to claim 1, wherein the catalyst is used in the form of a fixed-bed catalyst.

13. The process according to claim 1, wherein the reaction is carried out in a tube reactor at a temperature of from 30 to 110° C.

14. The process according to claim 13, wherein the temperature is increased during the course of the reaction.

15. The process according to claim 1, wherein the reaction is carried out in a tube reactor at a pressure of from 30 to 120 bar.

16. The process according to claim 1, wherein the space velocity over the catalyst is from 1 to 5 kg kg⁻¹h⁻¹.

17. A process for preparing 1,10-decanedial by isomerizing hydroformylation of mixtures comprising 1,7-octadiene and at least one further diolefin having at least one internal double bond.

18. A process for preparing 1,10-decanedial, wherein 1,7-octadiene is prepared by a process according to claim 1, and is hydroformylated.

19. The process according to claim 18, wherein 1,7-octadiene comprising further diolefins having at least one internal double bond is used.

20. The process according to claim 19, wherein 1,7-octadiene comprising 1,6-octadiene is used.

21. The process according to claim 19, wherein hydroformylation is carried out under isomerizing conditions.

22. A process for preparing muscone and/or its partially hydrogenated analogs, wherein decanedial is prepared by a process according to claim 1, and
a. is reacted with acetone in the presence of a base or a catalyst suitable for aldol reactions and, if appropriate, catalytically hydrogenated or is reacted with ethyl acetoacetate in the presence of a base, subsequently hydrolyzed, decarboxylated and, if appropriate, catalytically hydrogenated and
b. the reaction product is cyclised by intramolecular aldol condensation and, if appropriate, hydrogenated.

23. The process according to claim 22, wherein the aldol condensation of decanedial with acetone is carried out under hydrogenating conditions.
